(19) United States
(12) Patent Application Publication
(10) Pub. No.: US 2023/0183311 A1
(43) Pub. Date: Jun. 15, 2023

(54) POLYPEPTIDE USEFUL IN ADOPTIVE CELL THERAPY

(71) Applicant: QUELL THERAPEUTICS LIMITED, London, Greater London (GB)

(72) Inventors: Marc MARTINEZ-LLORDELLA, London, Greater London (GB); Simon BORNSCHEIN, Cremlingen (DE)

(21) Appl. No.: 17/926,374

(22) PCT Filed: May 26, 2021
(86) PCT No.: PCT/EP2021/064053

§ 371 Date: Nov. 18, 2022

(30) Foreign Application Priority Data
May 26, 2020 (GB) 2007842.4

(51) Int. Cl. C07K 14/705; C12N 15/63

(52) U.S. Cl. CPC C07K 14/70503; C12N 15/63; C07K 2319/03

(57) ABSTRACT

The present invention relates to a polypeptide comprising a sequence having the formula R1-L-R2-St wherein R1 and R2 are Rituximab-binding epitopes; St is a stalk sequence which, when the polypeptide is expressed at the surface of a target cell, causes the R1 and R2 epitopes to be projected from the cell surface; and L is a flexible linker sequence which connects the C terminus of R1 to the N terminus of R2. In particular, the linker sequence does not comprise a QBEnd10 binding epitope comprising the sequence set out in SEQ ID NO.1. The polypeptide functions as a suicide moiety which enables cells expressing the polypeptide to be deleted, and is useful in adoptive cell therapy. Also provided is a nucleic acid encoding such a polypeptide, a cell comprising such a nucleic acid and therapeutic uses thereof.

### FIELD OF THE INVENTION

The present invention relates to a polypeptide useful in adoptive cell therapy (ACT). The polypeptide comprises a suicide moiety, namely an epitope which enables cells expressing the polypeptide to be deleted. The polypeptide thus provides a means to provide a cell with a safety switch, which allows the cell to be “turned off”, or eliminated. The present invention also provides a nucleic acid encoding such a polypeptide, a cell comprising such a nucleic acid and therapeutic uses thereof.

### BACKGROUND TO THE INVENTION

Following early promise, adoptive cell therapy (ACT) is increasingly being used and tested in clinical application against malignant and infectious disease. T cells genetically engineered to recognise CD19 have been used to treat follicular lymphoma and ACT using autologous lymphocytes genetically-modified to express anti-tumour T cell receptors has been used to treat metastatic melanoma. The success of ACT in melanoma and EBV- associated malignancies spurred efforts to retarget effector T cells to treat other tumours, and T cells have been engineered to express T cell receptors (TCRs) or Chimeric antigen receptors (CARs) with new specificities.

CAR-modified T lymphocytes have been reported for immunotherapy of B-lineage malignancies (Kohn et al (2011) Mol. Ther. 19:432-438), and anti-GD2 CAR-transduced T cells for treatment of neuroblastoma (Pule et al (2008) Nat. Med. 14:1264-1270). Data showing efficacy has also been reported in clinical studies of CARs in adult lymphoma, and T-cells transduced with native T-cell receptors recognizing melanoma antigens have resulted in dramatic remissions in disseminated melanoma.

Other types of immune cells are also being used or proposed for use in ACT, including for example, NK cells, including NK cells engineered to express CARs. More recently, regulatory T cells (Tregs) have been developed for ACT. Tregs have immunosuppressive function. They act to control cytopathic immune responses and are essential for the maintenance of immunological tolerance. The suppressive properties of Tregs can be exploited therapeutically, for example to improve and/or prevent immune-mediated organ damage in inflammatory disorders, autoimmune diseases and in transplantation.

Increasing efficacy of adoptive immunotherapy has been associated with reports of serious adverse events. Acute adverse events, such as cytokine storms, have been reported after infusion of engineered T-cells. In addition, chronic adverse events have occurred and others have been predicted by animal models. For example, T-cells re-directed to carbonic anhydrase IX (CAIX), an antigen expressed by renal carcinoma, produced hepatotoxicity in several patients due to unexpected CAIX expression on biliary epithelium. Native T-cell receptor transfer studies against melanoma have resulted in vitiligo and iritis in patients due to expression of target antigen on skin and iris. A graft-versus host disease (GvHD)-like syndrome due to TCR cross-pairing has been reported in mice after native TCR transfer. A lymphoproliferative disorder has been reported in an animal model after adoptive transfer with some CARs which incorporate co-stimulation. Finally, the risk of vector insertional mutagenesis is always present. While acute toxicities can be addressed by cautious dosing, chronic toxicities are likely to be cell dose independent.

Since engineered T effector cells can expand and persist for years after administration, and in view of the ever present risk of an adverse event after patient administration of any immunotherapy, it is desirable to include a safety mechanism to allow selective deletion of adoptively infused T-cells and other immune cells in the face of toxicity.

Suicide genes enable selective deletion of transduced cells in vivo. Two suicide genes have been subjected to clinical testing: HSV-TK and iCasp9. Herpes Simplex Virus Thymidine kinase (HSV-TK) expression in T-cells confers susceptibility to ganciclovir. HSV-TK use is limited to clinical settings of profound immuuppression such as haploidentical bone marrow transplantation as this viral protein is highly immunogenic. Further, it precludes the use of Ganciclovir for cytomegalovirus treatment. Inducible Caspase 9 (iCasp9) can be activated by administration of a small molecule pharmaceutical (AP20187). Use of iCasp9 depends on availability of clinical grade AP20187. In addition, the use of an experimental small molecule in addition to genetically engineered cell product may cause regulatory issues.

Other suicide genes are being developed and EP-2836511B has reported a construct based on a minimal epitope from the antigen CD20, which is recognised by the lytic antibody Rituximab. Rituximab is an immunotherapeutic chimeric monoclonal antibody against the protein CD20, which is primarily found on the surface of B cells. When Ritiximab binds to CD20 it triggers cell death and thus it may be used to target and kill cells expressing CD20. Peptides which mimic the epitope recognised by Rituximab (so-called mimotopes) have been developed, and these were used in EP2836511 as a suicide moiety in a combined suicide-marker construct also comprising a CD34 minimal epitope as the marker moiety.

Specifically, EP-2836511B focused on providing both suicide and marker moieties within a single compact polypeptide, and to this aim developed a polypeptide, termed RQR8, represented by SEQ ID NO. 4 of EP-2836511. RQR8 comprises two cyclic peptide CD20 mimotopes (“R”) which flank a specific CD34 epitope (“Q”) having the sequence of SEQ ID NO. 1 herein (corresponding to SEQ ID NO. 2 of EP-2836511B), which is recognised by the monoclonal antibody QBEnd10. This is important as the QBEnd10 antibody is used in the Miltenyi CliniMACS magnetic cell selection system, which is widely used for isolation of cells in clinical settings. Accordingly, the inclusion of the Q epitope as a marker allows cells which have been modified to express this polypeptide readily to be selected using a commonly available selection system. Crucially, the R and Q epitopes in the polypeptide are separated from one another by spacer sequences (“S”) according to the general formula: St-R1-S1-Q-S2-R2. The spacer sequences S1 and S2, which need to have a combined length of at least 10 amino acids (they are 14 amino acids in the specific construct RQR8), in combination with Q, have been discussed as being important to keep the R1 and R2 epitopes at the correct distance, such that the polypeptide cannot bind both antigen binding sites of Rituximab simultaneously, ensuring that the polypeptide is capable of effectively inducing cell death. In particular, it is stated that the distance between R1 and R2 may be more than 76.57 Å. St is a stalk sequence which allows the R and Q epitopes to be projected from the cell surface when the polypeptide is expressed on a cell. In RQR8 the stalk sequence is from CD8.

The need for improved or alternative suicide constructs for use in ACT continues, including constructs which are not limited to use of the QBEnd10 CD34 marker system, and the present invention is directed to this need.

### SUMMARY OF THE INVENTION

In particular, in developing the present invention the present inventors have realised that the physical distance, or spacing, between the CD20 epitopes (the R epitopes) is not as critical or important as believed in EP-2836511B. In particular, the present inventors have determined that the prevention of binding of both R epitopes to the same Rituximab molecule may be achieved by focusing on the flexibility of the sequence which separates them, rather than solely on physical distance (i.e. the length of the sequence which separates them). Thus, the present inventors have shown that in fact functional suicide polypeptides may be produced in which the R epitopes are separated by much shorter sequences than those required in EP-2836511B. Further, by not including a marker between the R epitopes constraints on the design of the polypeptide may be removed, and a much wider range of different linker sequences may be used to link together and separate the R epitopes of the suicide polypeptide constructs. Such polypeptides may find utility in a wider range of cell modification protocols and applications than those limited to the use of the Miltenyi CliniMACS system. Particularly, the inventors have discovered that by increasing the flexibility of the sequence that separates the two R epitopes, the ability of the construct to induce cell lysis, particularly, the sensitivity of the construct to Rituximab or a biosimilar thereof, is also increased, and the invention further encompasses the development of improved constructs with high cell depletion ability. Specifically, the use of constructs with increased Rituximab sensitivity could result in a reduction in the amount of Rituximab required for administration to a patient in the instance of an adverse effect.

Accordingly, in a first aspect, the present invention provides a polypeptide comprising a sequence having the formula:

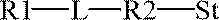

wherein
R1 and R2 are Rituximab-binding epitopes;
St is a stalk sequence which, when the polypeptide is expressed at the surface of a target cell, causes the R1 and R2 epitopes to be projected from the cell surface; and
L is a flexible linker sequence which connects the C terminus of R1 to the N terminus of R2 and which does not comprise a QBEnd10 binding epitope comprising the sequence set out in SEQ ID NO.1.

More particularly, L may be selected from:
(i) a flexible linker sequence having a length of no more than 25, preferably no more than 24, 23, 22 or 21 amino acids; and/or
(ii) a linker sequence which comprises at least 40% Gly or Gly and Ser residues; and/or
(iii) a linker sequence comprising Ser and/or Gly residues, and no more than 15 other amino acid residues, preferably no more than 14, 13, 12, 11, 10, 9, 8, 6, 7, 5, or 4 other amino acid residues; and/or
(iv) a linker sequence having an amino acid sequence wherein at least 80%, 90% or 100% of the amino acid residues are Ser, Gly, Thr, Ala, Lys, and Glu residues; and/or
(v) a linker sequence having an amino acid sequence which does not comprise any Pro residues.

Accordingly, alternatively defined, this aspect of the present invention may be seen to provide a polypeptide comprising a sequence having the formula:

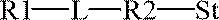

wherein
R1 and R2 are Rituximab-binding epitopes;
St is a stalk sequence which, when the polypeptide is expressed at the surface of a target cell, causes the R1 and R2 epitopes to be projected from the cell surface; and
L is a flexible linker sequence which connects the C terminus of R1 to the N terminus of R2 wherein L Is selected from:
   (i) a flexible linker which does not comprise the QBEnd10 binding epitope having the sequence set out in SEQ ID NO.1; and/or
   (i) a flexible linker sequence having a length of no more than 25, preferably no more than 24, 23, 22 or 21 amino acids; and/or
   (ii) a linker sequence which comprises at least 40% Gly or Gly and Ser residues; and/or
   (iii) a linker sequence comprising Ser and/or Gly residues, and no more than 15 other amino acid residues, preferably no more than 14, 13, 12, 11, 10, 9, 8, 6, 7, 5, or 4 other amino acid residues; and/or
   (iv) a linker sequence having an amino acid sequence wherein at least 80%, 90% or 100% of the amino acid residues are Ser, Gly, Thr, Ala, Lys, and Glu residues; and/or
   (v) a linker sequence having an amino acid sequence which does not comprise any Pro residues.

More particularly, the polypeptide of the invention may have the formula R1-L-R2-St.

The polypeptide may be co-expressed with a therapeutic transgene, such as a gene encoding a TCR or CAR.

In an embodiment, the Linker L does not contain a marker. However, it is not precluded that the polypeptide comprises a marker. In an embodiment the polypeptide may comprise a marker other than in L. In another embodiment the polypeptide does not contain a marker. In another embodiment the polypeptide may be co-expressed with a marker.

The polypeptide may comprise the sequence shown as SEQ ID NO. 27 or a variant thereof which has at least 80% identity with the sequence shown as SEQ ID NO.27 and which (i) binds Rituximab and (ii) when expressed on the surface of a cell, induces killing of the cell in the presence of Rituximab.

In a second aspect, the present invention provides a fusion protein which comprises a polypeptide of the invention as defined herein and a polypeptide fusion partner, e.g. a polypeptide of the invention of defined herein linked to a polypeptide fusion partner, optionally via a linker sequence. The fusion partner may be a protein of interest (POI).

The POI may be an antigen receptor, e.g. a chimeric receptor such as a chimeric antigen receptor (CAR), or a T cell receptor (TCR), or a marker.

The fusion protein may comprise a self-cleaving peptide between the polypeptide and the fusion partner, e.g. a protein of interest.

In one embodiment, the polypeptide of the invention may be comprised within the polypeptide fusion partner, e.g. the POI. In other words, the polypeptide may be fused, or linked, internally in the fusion partner. In this aspect, the polypeptide of the invention may, for example, be comprised within a CAR, e.g. within the extracellular domain of the CAR. A CAR may therefore comprise an extracellular domain, comprising an antigen targeting portion, e.g. a scFv and a polypeptide of the invention.

In a third aspect, the present invention provides a nucleic acid molecule comprising a nucleotide sequence capable of encoding a polypeptide or fusion protein according to the invention as defined herein.

In a fourth aspect, the present invention provides a vector which comprises a nucleic acid molecule of the invention as defined herein.

The vector may also comprise another coding sequence or other nucleotide sequence of interest. For example, it may comprise a nucleotide sequence which represents a transgene of interest, which may in one embodiment encode a protein of interest, e.g. a chimeric antigen receptor or a T-cell receptor or a marker.

In a fifth aspect, the present invention provides a cell which expresses a polypeptide of the invention as defined herein.

There is also provided a cell which comprises a nucleic acid molecule or a vector as defined herein.

The cell may co-express the polypeptide and a POI at the cell surface.

The cell may be an immune cell or a precursor therefor, such as a pluripotent stem cell (PSC) e.g. an iPSC, particularly a T cell, NK cell, dendritic cell or myeloid-derived suppressor cell (MDSC), e.g. a Treg cell, which includes such cells derived from a precursor, as well as primary cells and cell lines.

In a sixth aspect, the present invention provides a method for making a cell according to the fifth aspect of the invention which comprises the step of introducing into the cell (e.g. transducing or transfecting a cell with) a vector according to the fourth aspect of the invention.

In a seventh aspect, the present invention provides a method for deleting a cell according to the fifth aspect of the invention, which comprises the step of exposing the cells to an antibody having the binding specificity of Rituximab. In one aspect, the method may be an in vitro method.

Alternatively viewed, this aspect of the invention may comprise an antibody having the binding specificity of Rituximab for use in treating a subject to whom a cell of the invention as defined herein has been administered, to delete the cell.

Still further according to this aspect the invention provides a kit, or combination product, comprising (a) a polynucleotide, vector or cell of the invention as defined herein and (b) an antibody having the binding specificity of Rituximab. The kit or product may be for use in ACT. In particular, the kit or product may be for use in treating a subject by ACT using the cell or manufacturing a cell of the invention for use, and thereafter deleting the cell from the subject. The antibody may be administered to the subject following administration of the cell, for example after a period of time, or if the subject exhibits an unwanted or deleterious symptom or effect of the cell therapy.

In an embodiment the antibody may be Rituximab.

In an eighth aspect, the present invention provides a method for treating a disease in a subject, which comprises the step of administering to the subject a cell according to the fifth aspect of the invention. The subject may be a subject in need of the treatment and the cell may be administered in an amount effective for the treatment thereof.

Alternatively defined, this aspect may relate to a method of treating a subject by ACT, or a method of ACT of a subject.

The method may comprise the following steps:
(i) introducing into a sample of cells (e.g. a sample of cells isolated from a subject or obtained from a donor) a vector according to the fourth aspect of the invention (e.g. by transducing or transfecting the cells with the vector), and
(ii) administering the cells comprising the vector to the subject (e.g. in the case of autologous cells, returning the cells to the subject).

The method may comprise a further step of administering to the subject an antibody having the binding specificity of Rituximab.

In a ninth aspect, the present invention provides a cell according to the fifth aspect of the invention for use in therapy.

In a tenth aspect the present invention provides a cell according to the fifth aspect of the invention for use in therapy by adoptive cell transfer.

The method or the use may be for treating cancer or an infectious or neurodegenerative disease or for immunosuppression.

### DESCRIPTION OF THE FIGURES

FIG. 1: Illustration showing the Rituximab safety switch design (A). Two Rituximab based mimotopes were fused to a CD8 stalk sequence. The two Rituximab mimotopes were spaced with different linker sequences (B).

FIG. 2: The different Rituximab safety switches were co-expressed with an eGFP from a lentiviral vector. Here, Jurkat cells were transduced with the indicated constructs and A) eGFP expression and B) expression of the safety switch was assessed by flow cytometry.

FIG. 3: Complement dependent killing was assessed by culturing stably transduced cells with the different safety switches and culturing them in the presence of i) baby rabbit complement, ii) baby rabbit complement and Rituximab and iii) RPMI medium alone. After 4 hours percentage of killing was assessed by flow cytometry.

FIG. 4: Different Rituximab safety switches (RQR8, RR8 small (1xSGGGGS), RR8 large (3xSGGGGS) and Mock) were co-expressed with an eGFP from a lentiviral vector. Here, Jurkat cells were transduced with the indicated constructs and eGFP expression and expression of the safety switch was assessed by flow cytometry. Median Fluorescent Intensity (MFI) is shown.

FIG. 5: The sensitivity of the safety switches RQR8, RR8 small (1xSGGGGS) and RR8 large (3xSGGGGS) in stably transduced cells and mock transduced cells was examined by incubating the cells in the presence of i) baby rabbit complement and 100 ug/ml Rituximab, ii) baby rabbit complement and 5ug/ml Rituximab, iii) baby rabbit complement and 2.5 ug/ml Rituximab, iv) baby rabbit complement and 1.25 ug/ml Rituximab, v) baby rabbit complement and 0.625 ug/ml Rituximab, vi) baby rabbit complement and vii) RPMI medium alone. After incubation, percentage viability was analysed by FACS. FIG. 5A shows % live transduced cells in the CDC assay, and FIG. 5B shows % cell killing.

### DETAILED DESCRIPTION

The present invention provides a polypeptide which may be used as a suicide construct when expressed on the surface of a cell. This may be useful as a safety mechanism, or safety switch, which allows an administered cell to be deleted should the need arise, or indeed more generally, according to desire or need, for example once a cell has performed or completed its therapeutic effect, e.g. once a therapeutic transgene has been expressed.

The polypeptide comprises a suicide moiety. A suicide moiety possesses an inducible capacity to lead to cellular death. An example of a suicide moiety is a suicide protein, encoded by a suicide gene, which may be in included in a vector for expression of a desired transgene, which when expressed allows the cell to be deleted to turn off expression of the transgene.

In the polypeptide the suicide moiety comprises a minimal epitope based on the epitope from CD20 that is recognised by the antibody Rituximab. More particularly, the polypeptide comprises two CD20 epitopes R1 and R2 that are spaced apart by flexible linker L.

Cells expressing a polypeptide comprising this sequence can be selectively killed using the antibody Rituximab, or an antibody having the binding specificity of Rituximab. The suicide polypeptide is stably expressed on the cell surface after, for example, retroviral transduction of its encoding sequence. When the expressed polypeptide is exposed to or contacted with Rituximab, or an antibody with the same binding specificity, death of the cell ensues.

Retroviral transduction is a common way of introducing nucleic acids into mammalian cells, particular for therapy. However, retroviral vectors have packaging limits and generally it is desired to keep the size of introduced nucleic acids as small as possible. Although separate vectors may be used to introduce the suicide gene and desired transgene into a cell, it may be desired or convenient to introduce both the transgenes and the suicide gene in the same vector. By providing a polypeptide comprising a flexible linker to connect the two R epitopes the length of the polypeptide may be varied, and a short but flexible linker may be provided. This may allow greater latitude for the size of the transgene to be co-expressed with the polypeptide.

According to one aspect, or in one embodiment, the linker sequence L of the polypeptide of the invention does not comprise a QBEnd10-binding epitope comprising the amino acid sequence shown as SEQ ID NO. 1. Alternatively defined, the linker sequence L of polypeptide of the invention does not comprise a QBEnd10-binding epitope comprising the amino acid sequence shown as SEQ ID NO. 1 or a variant thereof which retains QBEnd10-binding activity. In other embodiments the polypeptide does not comprise a QBEnd10-binding epitope comprising the amino acid sequence shown as SEQ ID NO. 1 or a variant thereof which retains QBEnd10-binding activity.

SEQ ID NO.1 has the 16 amino acid sequence: ELPTQG
TFSNVSTNVS.

Antibody QBEnd10 is available from various sources including Abcam, ThermoFisher, Santa Cruz Biotechnology and Bio-Rad. Details of the antibody are available in EP3243838A1 and Chia-Yu Fan et al. Biochem Biophys Rep. 2017 Mar; 9: 51-60.

Further, in an embodiment the polypeptide, or the linker sequence L thereof, does not include, or comprise, an epitope derived from CD34. In another embodiment the polypeptide, or the linker sequence L thereof, does not include, or comprise, a minimal CD34 epitope.

A variant QBEnd10-binding epitope may comprise sequence modifications to the sequence of SEQ ID NO.1, subject to the modified sequence retaining at least 80% sequence identity. Deliberate amino acid substitutions may be made on the basis of similarity in polarity, charge, solubility, hydrophobicity, hydrophilicity, and/or the amphipathic nature of the residues as long as QBEnd10-binding activity of the epitope is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine. Generally, conservative substitutions may be made. The QBEnd10-binding epitope may, for example, contain 5 or fewer, 4 or fewer, 3 or fewer, 2 or fewer or 1 amino acid mutation(s) compared to the sequence shown as SEQ ID NO. 1. The QBEnd10-binding epitope may consist of the sequence shown as SEQ ID NO. 1 or a variant thereof which retains QBEnd10-binding activity.

The linker sequence of the polypeptides of the invention is a flexible linker sequence. Flexible linkers are a category of linker sequences well known and described in the art. Linker sequences are generally known as sequences which may be used to link, or join together, proteins or protein domains, to create for example fusion proteins or chimeric proteins, or multifunctional proteins or polypeptides. They can have different characteristics, and for example may be flexible, rigid or cleavable. Protein linkers are reviewed for example in Chen et al., 2013, Advanced Drug Delivery Reviews 65, 1357-1369, which compares the category of flexible linkers with those of rigid and cleavable linkers. Flexible linkers are also described in Klein et al., 2014, Protein Engineering Design and Selection, 27(10), 325-330; van Rosmalen et al., 2017, Biochemistry, 56,6565-6574; and Chichili et al., 2013, Protein Science, 22, 153-167.

A flexible linker is a linker which allows a degree of movement between the domains, or components, which are linked. They are generally composed of small non-polar (e.g. Gly) or polar (e.g. Ser or Thr) amino acid residues. The small size of the amino acids provides flexibility and allows for mobility of the connected parts (domains or components). The incorporation of polar amino acids can maintain the stability of the linker in aqueous environments by forming hydrogen bonds with water molecules.

The most commonly used flexible linkers have sequences primarily composed of Ser and Gly residues (so-called “GS linkers”). However, many other flexible linkers have also been described (see Chen et al,. 2013, supra, for example), which may contain additional amino acids such as Thr and/or Ala, and/or Lys and/or Glu which may improve solubility. Any flexible linker known and reported in the art may be used.

Although the length of the linker is not critical, it may in some embodiments be desirable to have a shorter linker sequence. For example, the linker sequence may have a length of no more than 25, preferably no more than 24, 23, 22 or 21 amino acids.

In other embodiments, a longer linker sequence may be desired, for example composed of, or comprising, multiple repeats of a GS domain.

In some embodiments the linker may be from any one of 2, 3, 4, 5 or 6 to any one of 24, 23, 22 or 21 amino acids in length. In other embodiments it may be from any one of 2, 3, 4, 5 or 6 to any one of 21, 20, 19, 18, 17, 16, or 15 amino acids in length. In other embodiments it may be intermediate between these ranges, from example from 6 to 21, 6 to 20, 7 to 20, 8-20, 9-20, 10-20, 8-18, 9-18, 10-18, 9-17, 10-17, 9-16, 10-16 etc. It may accordingly be in range made up from any of the integers listed above.

The use of GS linkers, or more particularly GS (“Gly-Ser”) domains in linkers, may allow the length of the linker readily to be varied by varying the number of GS domain repeats, and so such linkers represent one preferred class of linkers according to the present invention. However, flexible linkers are not limited to those based on “GS” repeats, and other linkers comprising Ser and Gly residues dispersed throughout the linker sequence have been reported, including in Chen et al., supra.

Accordingly, in one embodiment the linker sequence may comprise at least 40% Gly or Gly and Ser residues.

In another embodiment, the linker sequence may comprise Ser and/or Gly residues, and no more than 15 other amino acid residues, preferably no more than 14, 13, 12, 11, 10, 9, 8, 6, 7, 5, or 4 other amino acid residues. It will be understood than an “other” amino acid residue may be any amino acid which is not Ser or Gly.

Pro residues in linkers tend to confer rigidity and so in one embodiment the linker sequence does not comprise any Pro residues. However, this is not absolute, as depending on the sequence context, a flexible linker sequence may contain one or more Pro residues.

In one preferred embodiment, the linker sequence comprises at least one Gly-Ser domain composed solely of Ser and Gly residues. In such an embodiment, the linker may contain no more than 15 other amino acid residues, preferably no more than 14, 13, 12, 11, 10, 9, 8, 6, 7, 5, or 4 other amino acid residues.

The Gly-Ser domain may have the formula:

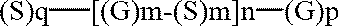

wherein q is 0 or 1; m is an integer from 1-8; n is an integer of at least 1 (e.g. from 1 to 8, or more particularly 1 to 6); and p is 0 or an integer from 1 to 3.

More particularly, the Gly-Ser domain may have the formula:

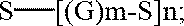

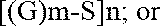

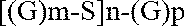

wherein m is an integer from 2-8 (for example 3-4); n is an integer of at least 1 (for example from 1 to 8, or more particularly 1 to 6); and p is 0 or an integer from 1 to 3.

In a representative example, the Gly-Ser domain may have the formula:

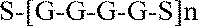

wherein n is an integer of at least one (preferably 1 to 8, or 1-6, 1-5, 1-4, or 1-3). In the formula above, the sequence GGGGS is SEQ ID NO. 31.

A linker sequence may be composed solely of, or may consist of, one or more Gly-Ser domains as described or defined above. However, as noted above, in another embodiment, the linker sequence may comprise one or more Gly-Ser domains, and additional amino acids. The additional amino acids may be at one or both ends of a Gly-Ser domain, or at one or both ends of a stretch of repeating Gly-Ser domains. Thus, the additional amino acid, which may be other amino acids, may lie at one or both ends of the linker sequence, e.g. they may flank the Gly-Ser domain(s). In other embodiments, the additional amino acids may lie between Gly-Ser domains. For example, two Gly-Ser domains may flank a stretch of other amino acids in the linker sequence. Further, as also noted above, in other linkers, GS domains need not be repeated, and G and/or S residues, or a short domain such as GS, may simply be distributed along the length or the sequence, for example as shown in SEQ ID NO. 41 below.

Representative exemplary linker sequences are listed below:

ETSGGGGSRL (SEQ ID NO. 32)
SGGGGSGGGGSGGGGS ((SEQ ID NO. 33)
S(GGGGS)₁₋₅ (where GGGGS is SEQ ID NO. 31)
(GGGGS)₁₋₅ (where GGGGS is SEQ ID NO. 31)
S(GGGS)₁₋₅ (where GGGS is SEQ ID NO. 34)
(GGGS)₁₋₅ (where GGGS is SEQ ID NO. 34)
S(GGGGGS)₁₋₅ (where GGGGGS is SEQ ID NO. 35)
(GGGGGS)₁₋₅ (where GGGGGS is SEQ ID NO. 35)
S(GGGGGGS)₁₋₅ (where GGGGGGS is SEQ ID NO. 36)
(GGGGGGS)₁₋₅ (where GGGGGGS is SEQ ID NO. 36)
G₆ (SEQ ID NO. 37)
G₈ (SEQ ID NO. 38)
KESGSVSSEQLAQFRSLD (SEQ ID NO.39)
EGKSSGSGSESKST (SEQ ID NO.40)
GSAGSAAGSGEF (SEQ ID NO.41)
SGGGGSAGSAAGSGEF (SEQ ID NO.42)
SGGGLLLLLLLLGGGS (SEQ ID NO.43)
SGGGAAAAAAAAGGGS (SEQ ID NO.44)
SGGGAAAAAAAAAAAAAAAAGGGS (SEQ ID NO.45)
SGALGGLALAGLLLAGLGLGAAGS (SEQ ID NO.46)
SLSLSPGGGGGPAR (SEQ ID NO.47)
SLSLSPGGGGGPARSLSLSPGGGGG (SEQ ID NO.48)
GSSGSS (SEQ ID NO.49)
GSSSSSS (SEQ ID NO.50)
GGSSSS (SEQ ID NO.51)
GSSSSS (SEQ ID NO.52)
SGGGGS (SEQ ID NO. 53.

In the polypeptides the function of the linker is to connect R1 to R2. The linker connects R1 and R2 directly, that is the C-terminus of R1 to the N-terminus of R2. The polypeptide does not contain any other component or sequence between R1 and R2 other than the linker sequence L. It will be understood that since the polypeptide is to be expressed on the surface of the cell and since R1 is connected to R2 such that both R1 and R2 are to be expressed on the surface of the cell, the linker L is not a cleavable linker.

In an embodiment the linker does not perform any other function, or does not comprise any other functional component or sequence. For example, the linker sequence does not possess, or does not comprise any sequence which has, a biological activity. In an embodiment, the linker does not comprise a marker sequence.

Although the linker sequences of the polypeptides of the invention, as defined above, are flexible sequences, the present disclosure includes also other polypeptides, including those which comprise linkers which are not flexible, and/or which do not meet the definitions and requirements set out above.

Thus, in other aspects a polypeptide is provided of formula

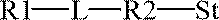

wherein
R1 and R2 are Rituximab-binding epitopes;
St is a stalk sequence which, when the polypeptide is expressed at the surface of a target cell, causes the R1 and R2 epitopes to be projected from the cell surface; and
L is a linker sequence which connects the C terminus of R1 to the N terminus of R2 and which (i) does not comprise a QBEnd10 binding epitope comprising the sequence set out in SEQ ID NO.1 and/or (ii) has a length of no more than 25, preferably no more than 24, 23, 22 or 21 amino acids.
R1, R2 and St may be as defined and described elsewhere herein. The Linker L may be any linker sequence, that is a linker sequence having any amino acid sequence subject to the constraints (i) and (ii) above (and that the linker sequence is not cleavable).

Examples of such linker sequences include:

SGGGSNVSTNVSPAKPTTTA (SEQ ID NO. 64)
SGGGSELPTQGTFSNVSTNA (SEQ ID NO. 65)
EAAAKEAAAKEAAAKEAAAK (SEQ ID NO. 66)
GGGGSEAAAKEAAAKSGGGS (SEQ ID NO. 67)
EAAAKEAAAKEAAAK (SEQ ID NO. 68)
GGLKNKAQQAAFYIGG (SEQ ID NO. 69)
LCKNKAQQAAFYCI (SEQ ID NO. 70)
KCLNDAQAAAEECI (SEQ ID NO. 71)
GGGLKNKAQQAAFYIGGG (SEQ ID NO. 72)
EAAAKEAAAKEAAAKEAAAEAAAKE (SEQ ID NO. 73)
GGGSEAAAKEAAAKEAAAKEGGGS (SEQ ID NO. 74).

A Rituximab-binding epitope is an amino acid sequence which binds to the antibody Rituximab, or an antibody which has the binding specificity of Rituximab, in other words an antibody which binds to the same natural epitope as does Rituximab. Rituximab is a chimeric mouse/human monoclonal kappa IgG1 antibody which binds human CD20. The Rituximab-binding epitope sequence from CD20 is CEPANPSEKNSPSTQYC (SEQ ID NO. 29).

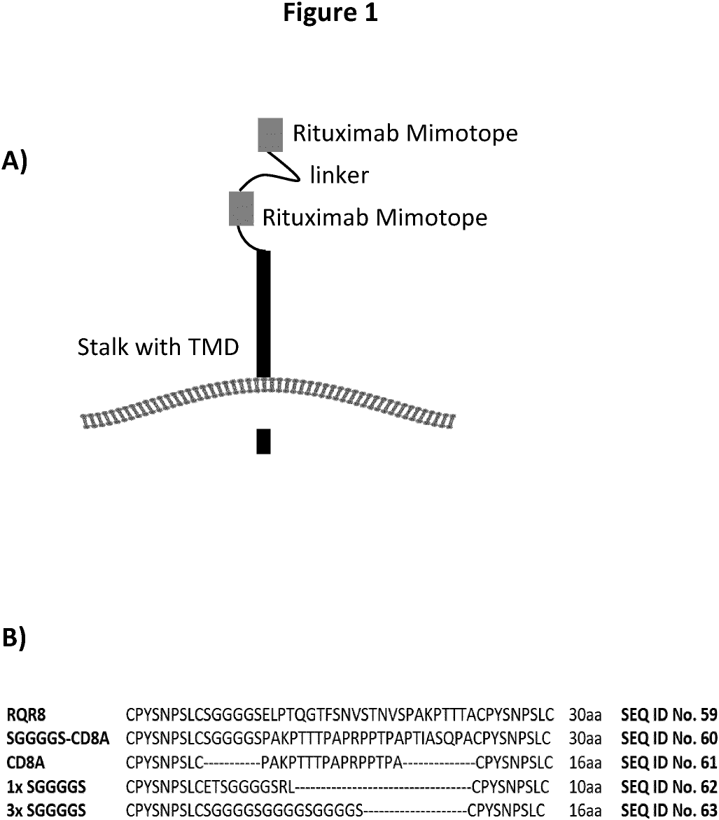

Rituximab was first described in EP0669836 (hybridoma) and the heavy and light chain sequences are given in EP2000149 (see also Wang et al.,Analyst, 2013, 138, 3058, which gives the heavy and light chain sequences in FIG. 1 thereof and Rituximab- CAS 17422-31-7, catolog number: B0084-061043, BOC Sciences). Reference may also be made to US 2009/0285795 A1, EP 1633398 A2, and WO 2005/000898. Rituximab and biosimilars thereof are widely available from various commercial sources around the world.

R1 and R2 may thus be any peptide which binds to, or in other words which is capable of binding to, Rituximab. As well as the natural epitope in the context of CD20, various peptides are known, and have been reported, which bind to Rituximab, or more particularly which mimic the natural epitope. R1 and R2 may accordingly be a mimotope of the Rituximab epitope.

Such mimotopes are described for example in Perosa et al (2007, J. Immunol 179:7967-7974) which discloses a series of cysteine-constrained 7-mer cyclic peptides, which bear the antigenic motif recognised by Rituximab but have different motif-surrounding amino acids. Perosa describe eleven peptides with SEQ ID NO.s 15 to 25, as shown in Table 1 below. In the Table the amino acids flanking the motif are shown in lower case, and the motif is shown in upper case. It has been determined that the initial amino acid “a” may be removed from the peptide and a functional epitope (or mimotope) may be retained. Peptides of SEQ ID NOS. 4 to 14 lacking the initial “a” are also shown in Table 1.

TABLE 1

| | | |
|---|---|---|
| Perosa peptide designation | Sequence | Modified sequence |
| R15-C | acPYANPSLc (SEQ ID NO. 15) | cPYANPSLc (SEQ ID NO. 4) |
| R3-C | acPYSNPSLc (SEQ ID NO. 16) | cPYSNPSLc (SEQ ID NO. 5) |
| R7-C | acPFANPSTc (SEQ ID NO. 17) | cPFANPSTc (SEQ ID NO. 6) |
| R8-, R12-, R18-C | acNFSNPSLc (SEQ ID NO. 18) | cNFSNPSLc (SEQ ID NO. 7) |
| R14-C | acPFSNPSMc (SEQ ID NO. 19) | cPFSNPSMc (SEQ ID NO. 8) |
| R16-C | acSWANPSQc (SEQ ID NO. 20) | cSWANPSQc (SEQ ID NO. 9) |
| R17-C | acMFSNPSLc (SEQ ID NO. 21) | cMFSNPSLc (SEQ ID NO. 10) |
| R19-C | acPFANPSMc (SEQ ID NO. 22) | cPFANPSMc (SEQ ID NO. 11) |
| R2-C | acWASNPSLc (SEQ ID NO. 23) | cWASNPSLc (SEQ ID NO. 12) |
| R10-C | acEHSNPSLc (SEQ ID NO. 24) | cEHSNPSLc (SEQ ID NO. 13) |
| R13-C | acWAANPSMc (SEQ ID NO. 25) | cWAANPSMc (SEQ ID NO. 14) |

A circular (or cyclic) mimotope of the Rituximab epitope which may be used as R1 and/or R2 according to the invention may be represented by the consensus amino acid sequence of SEQ ID NO. 2:

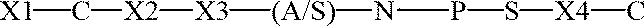

wherein X1 is A or absent, and X2, X3 and X4 are any amino acid.

More particularly, X2 may be an amino acid selected from P, N, S,M, W or E; X3 may be an amino acid selected from Y, F, W,A, or H; and X4 may be an amino acid selected from L, T, M or Q.

Non-circular (or non-cyclic) peptide mimotopes of the Rituximab epitope have also been developed. Li et al (2006 Cell Immunol 239:136-43) also describe mimotopes of Rituximab, including a peptide with the sequence QDKLTQWPKWLE (SEQ ID NO. 3).

The polypeptide may comprise Rituximab-binding epitopes R1 and R2 which each independently comprise an amino acid sequence selected from the group consisting of SEQ ID NO. 2, 3, or 4 to 25, or a variant thereof which retains Rituximab-binding activity.

The two epitopes R1 and R2 may be the same or different. In one embodiment they are the same. In another embodiment they are different.

In an embodiment, R1 and R2 each consist essentially of, or alternatively each consist, of an amino acid sequence selected from the group consisting of SEQ ID NO. 2 to 25, or a variant thereof which retains Rituximab-binding activity

In a representative embodiment, the polypeptide may comprise Rituximab-binding epitopes R1 and R2 comprising, consisting essentially of, or consisting of, the amino acid sequence shown as SEQ ID NO. 5 or 16 or a variant thereof which retains Rituximab-binding activity.

In an embodiment R1 consists of, consists essentially of, or comprises SEQ ID NO. 16 and R2 consists of, consists essentially of, or comprises SEQ ID NO. 5.

A variant Rituximab-binding epitope may be based on the sequence selected from the group consisting of SEQ ID NOs. 3-25 but comprises one or more amino acid mutations, such as amino acid insertions, substitutions or deletions, relative to the sequence, provided that the epitope retains Rituximab-binding activity. In particular, the sequence may be truncated at one or both terminal ends by, for example, one or two amino acids.

Deliberate amino acid substitutions may be made on the basis of similarity in polarity, charge, solubility, hydrophobicity, hydrophilicity, and/or the amphipathic nature of the residues as long as Rituximab-binding activity of the epitope is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to Table 2 below:

TABLE 2

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Amino acids in the same block in the second column and in the same line in the third column may be substituted for each other:

The Rituximab-binding epitope may, for example, contain 3 or fewer, 2 or fewer or 1 amino acid mutation(s) compared to the sequence selected from the group consisting of SEQ ID NOS. 3-25.

A variant of a Rituximab-binding epitope may comprise or consist of an amino acid sequence having at least 75% sequence identity to any one of SEQ ID NOS. 3 to 25, more particularly at least 80, 85, 90, 95, 96, 97, 98 or 99% sequence identity thereto.

Where two identical (or similar) Rituximab-binding amino acid sequences are used, it may be advantageous to use different nucleotide sequences to encode the two R epitopes. In many expression systems, homologous sequences can result in undesired recombination events.

Using the degeneracy of the genetic code, alternative codons may be used to achieve nucleotide sequence variation without altering the protein sequence thereby preventing homologous recombination events.

The polypeptide comprises a stalk sequence (St) which, when the polypeptide is expressed at the surface of a target cell, causes the R and Q epitopes to be projected away from the surface of the target cell.

The stalk sequence causes the R and Q epitopes to be sufficiently distanced from the cell surface to facilitate binding of, for example, Rituximab or an equivalent antibody.

The stalk sequence elevates the epitopes from the cell surface.

The stalk sequence may be a substantially linear amino acid sequence. The stalk sequence may be sufficiently long to distance the R and Q epitopes form the surface of the target cell but not so long that its encoding sequence compromises vector packaging and transduction efficiency. The stalk sequence may, for example be between 30 and 100 amino acids in length. The stalk sequence may be approximately 40-50 amino acids in length.

The stalk sequence may be highly glycosylated.

The stalk sequence may comprise a linker sequence which links or connects it to the epitope R2 in the formula above.

A wide range of proteins are known which are expressed on the surface of mammalian cells and which can be used to provide, or as the basis for, a stalk sequence herein. Such surface-expressed proteins comprise natural sequences which can be used as, or to derive, a stalk sequence. For example, the extracellular domain (ECD) of such a protein may be used as a stalk sequence, or the extracellular and transmembrane (TM) domains, or the extracellular and transmembrane domains (ECD and TMD) with an intracellular domain (ICD) which may serve as an intracellular anchor to hold the stalk in the membrane and allow it to project from the cell surface.

Such proteins include CD27, CD28, CD3 epsilon, CD3z, CD45, CD4, CD5, CD8, CD9, CD16, CD18, CD22, CD33, CD37, CD64, CD80, CD86, CD134, CD137, CD152, CD154, CD278, CD279, IgG1, or IgG2.

Thus, the stalk sequence St may comprise an optional linker sequence which connects it to R2, an extracellular domain, an optional transmembrane domain, and an optional intracellular domain.

In an embodiment the stalk sequence may comprise a linker sequence which connects it to R2, an extracellular domain, a transmembrane domain, and an intracellular domain

The stalk sequence, or the extracellular domain thereof, may comprise or be approximately equivalent in length to the sequence:

PAPRPPTPAPTIASQPLSLRPEACRPAAGGAVHTRGLDFACD (SEQ ID
NO. 30),

which is the extracellular sequence from CD8.

As noted above, the stalk sequence may additionally comprise a transmembrane domain, optionally together with an intracellular domain, which may serve as an intracellular anchor sequence. The transmembrane domain and intracellular domain may be derived from the same protein as extracellular domain or it/they may be derived from a different protein. The transmembrane domain and intracellular domain may be derivable from CD8.

The stalk sequence St may comprise an extracellular stalk sequence, a transmembrane domain, and an intracellular domain derived from CD8.

A CD8 stalk sequence which comprises a transmembrane domain and an intracellular anchor may have the following sequence:

PAPRPPTPAPTIASQPLSLRPEACRPAAGGAVHTRGLDFACDIYIWAPLA
GTCGVLLLSLVITLYCNHRNRRRVCKCPRPVV(SEQ ID NO. 26)

or a sequence which hast at least 75% particularly at least 80, 85, 90, 95, 96, 97, 98 or 99% sequence identity thereto.

Within this sequence, the underlined portion corresponds to the CD8a stalk; the central portion corresponds to the transmembrane domain; and the portion in bold corresponds to the intracellular domain.

The linker sequence in a stalk sequence may be a linker as described above. In particular, it may be a linker sequence which comprises or consists of Ser (S) and/or Gly (G) residues. The linker sequence(s) may be substantially linear. In the context of the stalk, the linker sequence may be a shorter sequence. For example, the linker sequence(s) may have the general formula:

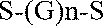

where n is a number between 2 and 8.

The linker may comprise or consist of the sequence SGGGGS (SEQ ID NO. 53).

Representative exemplary embodiments of the polypeptide of the invention include polypeptides comprising Rituximab binding epitopes of SEQ ID NO. 5 and/or 16 linked via a linker to a stalk sequence comprising extracellular, transmembrane and intracellular sequences derived from CD8. In particular, the stalk sequence may have the sequence of SEQ ID NO. 26. The linker L between R1 and R2 may be any of the linkers of or based on SEQ ID NOS. 32-53 above. Particularly, the linker L may have the sequence set out in SEQ ID NOS. 32 or 33 above.

The stalk sequence may comprise a linker sequence which connects to R2. The linker sequence in the stalk may be SGGGS (SEQ ID NO. 53)

Accordingly, the polypeptide of the invention may comprise or consist of the amino acid sequence shown as SEQ ID. NO. 27, 28, 78 or 79, or a sequence having at least 75% , particularly at least 80, 85, 90, 95, 96, 97, 98 or 99% sequence identity thereto.

CPYSNPSLCETSGGGGSRLCPYSNPSLCSGGGGSPAPRPPTPAPTIASQP
LSLRPEACRPAAGGAVHTRGLDFACDIYIWAPLAGTCGVLLLSLVITLYC
NHRNRRRVCKCPRPVV (SEQ IDNO. 27)
CPYSNPSLCSGGGGSGGGGSGGGGSCPYSNPSLCSGGGGSPAPRPPTPAP
TIASQPLSLRPEACRPAAGGAVHTRGLDFACDIYIWAPLAGTCGVLLLSL
VITLYCNHRNRRRVCKCPRPVV (SEQ ID NO. 28)
ACPYSNPSLCETSGGGGSRLCPYSNPSLCSGGGGSPAPRPPTPAPTIASQ
PLSLRPEACRPAAGGAVHTRGLDFACDIYIWAPLAGTCGVLLLSLVITLY
CNHRNRRRVCKCPRPVV (SEQID NO.78)
ACPYSNPSLCSGGGGSGGGGSGGGGSCPYSNPSLCSGGGGSPAPRPPTPA
PTIASQPLSLRPEACRPAAGGAVHTRGLDFACDIYIWAPLAGTCGVLLLSL
VITLYCNHRNRRRVCKCPRPVV (SEQ ID NO. 79)

The polypeptide may also comprise, or be expressed with, a signal peptide at the amino terminus. A number of different signal sequences are known and reported in the art and it would be a matter of routine to select a signal peptide. The signal peptide may, for example, comprise or consist of the sequence shown as SEQ ID NO. 54 or SEQ ID NO. 80.

MGTSLLCWMALCLLGADHADA (SEQ ID NO. 54)

MGTSLLCWMALCLLGADHAD (SEQ ID NO. 80)

A polypeptide comprising a signal peptide of SEQ ID NO.54 and the amino acid sequence of SEQ ID NO. 27 is represented by SEQ ID NO. 55. It will also be seen that SEQ ID NO. 55 represents a polypeptide comprising a signal peptide of SEQ ID NO. 80 and the amino acid sequence of SEQ ID NO. 78. A polypeptide comprising a signal peptide of SEQ ID NO.54 and the amino acid sequence of SEQ ID NO. 28 is represented by SEQ ID NO. 56. It will also be seen that SEQ ID NO. 56 represents a polypeptide comprising a signal peptide of SEQ ID NO. 80 and the amino acid sequence of SEQ ID NO. 79.

Once the polypeptide is expressed by the target cell (that is the cell into which a nucleic acid molecule comprising a nucleotide sequence encoding the polypeptide is introduced), the signal peptide is cleaved off, resulting in the mature polypeptide product.

The polypeptide of the invention may comprise or consist of a variant of the sequence shown as SEQ ID NO. 27 or 28, or SEQ ID NO. 78 or 79, which has at least 75% (e.g. at least 80%, 85%, 90% or 95%) identity with the sequence shown as SEQ ID NO. 27 or 28 or SEQ ID NO. 78 or 79, as long as it retains the functional activity of the SEQ ID NO. 27 or 28 or SEQ ID NO. 78 or 79 polypeptide. For example, the variant sequence should (i) bind Rituximab and (ii) when expressed on the surface of a cell, induce killing of the cell in the presence of Rituximab.

Homology comparisons may be conducted by eye or with the aid of readily available sequence comparison programs, such as the GCG Wisconsin Bestfit package.

In an embodiment the polypeptide consists only of the elements R1, L, R2 and St as set out and described above. In one embodiment does not comprise a marker sequence. However, in other embodiments the polypeptide may additionally comprise a marker sequence. However, any such marker sequence cannot lie as an additional element to L between R1 and R2. By way of example, a marker sequence may be included the stalk sequence, or may be introduced between the stalk and R2.

In other embodiments the polypeptide may be linked or coupled to other moieties.

The polypeptide may be in the form of a fusion protein, in which the polypeptide is fused to or within, or linked to, or comprised within a polypeptide fusion partner. A fusion partner is a separate, or second, polypeptide which does not occur with any component of the first polypeptide, that is the polypeptide of the invention, in nature. The fusion partner can be a second polypeptide which confers a desired property or function on the polypeptide. For example, it may be a marker, or may comprise a marker sequence. The fusion partner may be a protein of interest (POI). The fusion partner may be linked to the polypeptide by a linker sequence. The linker sequence in this context may be any known or desired linker sequence which is appropriate and functional to link the protein to the fusion partner. This may include any linker sequence discussed above. Further, the linker sequence may be a cleavable linker sequence.

The fusion protein may comprise a self-cleaving peptide between the polypeptide and the fusion partner (e.g. the protein of interest). Such a self-cleaving peptide would allow co-expression of the polypeptide and the POI within the target cell, followed by cleavage so that the polypeptide and POI are expressed as separate proteins at the cell surface. For example, the fusion protein may comprise the foot-and-mouth disease self-cleaving 2A peptide. Options for self-cleaving peptides are known in the art.

The protein of interest may be a molecule for expression at the surface of a target cell. That is, it may be a polypeptide that it is desired to express on the surface of a cell along with the polypeptide of the invention. The POI may exert a therapeutic or prophylactic effect when the target cell is in vivo.

The POI may be an antigen receptor. For example, it may be a chimeric receptor or a T cell receptor (TCR). A chimeric receptor may be a chimeric antigen receptor (CAR).

Chimeric antigen receptors are generated by joining an antigen-recognising domain (ectodomain) to the transmembrane and intracellular portion of a signalling molecule (endodomain). The ectodomain is most commonly derived from antibody variable chains (for example a scFv), but may also be generated from T-cell receptor variable domains or other molecules, such as receptors for ligands or other binding molecules. The endodomain may comprise at least the intracellular portion of CD3-ζ. The endodomain may comprise a CD28-OX40-CD3ζ tripartite cytoplasmic domain. Various different transmembrane and combinations of intracellular signalling and co-stimulatory domains are known in the art.

The POI may be a receptor, e.g. a CAR or TCR, with specificity for an antigen associated with disease or with an unwanted clinical condition, for example cancer, infection, a neurodegenerative condition, or an unwanted immune response, e.g. an autoimmune or allergic response, or GvHD or transplant rejection. ACT with cells expressing an antigen receptor may further be used to induce tolerance, promote tissue repair and/or tissue regeneration, or to ameliorate chronic inflammation, e.g. secondary to metabolic disorders (see WO 2020/044055, for example).

The receptor may have specificity for a tumour-associated antigen, (i.e. a protein which is expressed or overexpressed on cancer cells). Such proteins include ERBB2 (HER-2/neu), which is overexpressed in 15-20% of breast cancer patients and is associated with more aggressive disease; CD19, which is expressed on most B-cell malignancies; carboxy-anhydrase-IX, which is frequently overexpressed in renal cell carcinoma; GD2, which is expressed by neuroblastoma cells; p53; MART-1 (DMF5); gp100:154; NY-ESO-1; and CEA.

To treat or prevent an immune disorder or an unwanted immune response, or induce tolerance etc., a CAR may be expressed in a Treg cell, wherein the CAR may for example comprise an endodomain which comprises a STAT5 association motif and a JAK1- and/or a JAK2-binding motif, as described in WO 2020/044055. CARs for use in the prevention or treatment of organ transplantation rejection e.g. liver or kidney transplantation rejection, may have specificity for HLA, e.g. HLA-A2 which is commonly mismatched between transplant donors and recipients.

The second aspect of the invention relates to a nucleic acid molecule comprising a nucleotide sequence capable of encoding a polypeptide or fusion protein of the invention.

The nucleic acid, when expressed by a target cell, causes the encoded polypeptide to be expressed at the cell-surface of the target cell. Where the nucleic acid encodes both the polypeptide and POI (for example as a fusion protein), it may cause both the polypeptide of the invention and the POI to be expressed at the surface of the target cell.

The nucleic acid molecule may be RNA or DNA, such as cDNA.

Nucleotide sequences encoding representative polypeptides of SEQ ID NOS. 55 and 56 are shown in SEQ ID NOS. 57 and 58 respectively. Accordingly, a nucleic acid molecule of the invention may comprise a nucleotide sequence as shown in SEQ ID NO. 57 or 58, or a nucleotide sequence having a least 70% (e.g. at least 75, 80, 85, 90 or 95 %) sequence identity with SEQ ID NO. 57 or 58.

The present invention also provides a vector which comprises a nucleic acid molecule of the present invention. The vector may also comprise a transgene of interest, that is a nucleotide sequence encoding or providing an element of interest. Such a transgene may be a gene encoding a POI.

The vector should be capable of transfecting or transducing a target cell (e.g. either alone or in the presence of another reagent/entity), such that they express the polypeptide of the invention and optionally a protein of interest.

The vector may be a non-viral vector such as a plasmid. Plasmids may be transfected into cells using any well-known method of the art, e.g. using calcium phosphate, liposomes or cell penetrating peptides (e.g. amphipathic cell penetrating peptides).

The vector may be a viral vector, such as a retroviral vector, e.g. a lentiviral vector or a gamma retroviral vector.

Vectors suitable for delivering nucleic acids for expression in mammalian cells are well known in the art and any such vector may be used. Vectors may comprise one or more regulatory elements, e.g. a promoter.

Delivery systems for introducing a nucleic acid to a cell are also known and used in the art which do not rely on vectors, including for example systems based on transposons, CRISPR/TALEN delivery and mRNA delivery. Any such system can be used to deliver a nucleic acid molecule according to the present invention. Thus, the present invention also provides a recombinant construct for delivery into a cell, said construct comprising a nucleic acid molecule of the invention as defined and described herein. Such a construct may include another (e.g. a further or second) nucleic acid molecule or nucleotide sequence or genetic element, which enables or facilitates delivery of the nucleic acid molecule to a cell.

The vector or recombinant construct may comprise a nucleic acid encoding the polypeptide and a nucleic acid comprising the POI as separate entities, or as a single nucleotide sequence. If they are present as a single nucleotide sequence they may comprise one or more internal ribosome entry site (IRES) sequences or other translational coupling sequences between the two encoding portions to enable the downstream sequence to be translated. A cleavage site such as a 2A cleavage site (e.g. T2A or P2A) may be encoded by a nucleic acid or vector or recombinant construct of the invention, particularly between the polypeptide of the invention and any POI.

The present invention also provides a cell which expresses a polypeptide according to the first aspect of the invention. The cell may express the polypeptide or co-express the polypeptide and a POI at the cell surface. The cell may be referred to as a target cell.

The present invention also provides a cell which comprises a nucleic acid molecule capable of encoding a polypeptide according to the first aspect of the invention.

The cell may be a cell into which a nucleic acid molecule or vector or recombinant construct as described herein has been introduced. The cell may have been transduced or transfected with a vector or recombinant construct according to the invention.

The cell may be suitable for adoptive cell therapy.

The cell may be an immune cell, or a precursor therefor. A precursor cell may also be termed a progenitor cell, and the two terms are used synonymously herein. Representative immune cells thus include T-cells in particular cytotoxic T-cells (CTLs; CD8+ T-cells), helper T-cells (HTLs; CD4+ T-cells) and regulatory T cells (Tregs). Other populations of T-cells are also useful herein, for example naive T-cells and memory T-cells. Other immune cells include NK cells, NKT cells, dendritic cells, MDSC, neutrophils, and macrophages. Precursors of immune cells include pluripotent stem cells, e.g. induced PSC (iPSC), or more committed progenitors including multipotent stem cells, or cells which are committed to a lineage. Precursor cells can be induced to differentiate into immune cells in vivo or in vitro. In one aspect, a precursor cell may be a somatic cell which is capable of being transdifferentiated to an immune cell of interest.

Most notably, the immune cell may be an NK cell, a dendritic cell, a MDSC, or a T cell, such as a cytotoxic T lymphocyte (CTL) or a Treg cell.

The T cell may have an existing specificity. For example, it may be an Epstein-Barr virus (EBV)-specific T cell. Alternatively, the T cell may have a redirected specificity, for example, by introduction of an exogenous or heterologous TCR or a chimeric receptor, e.g. CAR.

In a preferred embodiment the immune cell is a Treg cell. “Regulatory T cells (Treg) or T regulatory cells” are immune cells with immunosuppressive function that control cytopathic immune responses and are essential for the maintenance of immunological tolerance. As used herein, the term Treg refers to a T cell with immunosuppressive function.

Suitably, immunosuppressive function may refer to the ability of the Treg to reduce or inhibit one or more of a number of physiological and cellular effects facilitated by the immune system in response to a stimulus such as a pathogen, an alloantigen, or an autoantigen. Examples of such effects include increased proliferation of conventional T cell (Tconv) and secretion of proinflammatory cytokines. Any such effects may be used as indicators of the strength of an immune response. A relatively weaker immune response by Tconv in the presence of Tregs would indicate an ability of the Treg to suppress immune responses. For example, a relative decrease in cytokine secretion would be indicative of a weaker immune response, and thus indicative of the ability of Tregs to suppress immune responses. Tregs can also suppress immune responses by modulating the expression of co-stimulatory molecules on antigen presenting cells (APCs), such as B cells, dendritic cells and macrophages. Expression levels of CD80 and CD86 can be used to assess suppression potency of activated Tregs in vitro after co-culture.

Assays are known in the art for measuring indicators of immune response strength, and thereby the suppressive ability of Tregs. In particular, antigen-specific Tconv cells may be co-cultured with Tregs, and a peptide of the corresponding antigen added to the co-culture to stimulate a response from the Tconv cells. The degree of proliferation of the Tconv cells and/or the quantity of the cytokine IL-2 they secrete in response to addition of the peptide may be used as indicators of the suppressive abilities of the co-cultured Tregs. Antigen-specific Tconv cells co-cultured with Tregs as described herein may proliferate 5%, 10%, 20%, 30%, 40%, 50%, 60%, 70%, 90%, 95% or 99% less than the same Tconv cells cultured in the absence of Tregs as described herein.

Antigen-specific Tconv cells co-cultured with Tregs may express at least 10%, at least 20%, at least 30%, at least 40%, at least 50%, or at least 60% less effector cytokine than corresponding Tconv cells cultured in the absence of Tregs. The effector cytokine may be selected from IL-2, IL-17, TNFα, GM-CSF, IFN-y, IL-4, IL-5, IL-9, IL-10 and IL-13. Suitably the effector cytokine may be selected from IL-2, IL-17, TNFα, GM-CSF and IFN-y.

Several different subpopulations of Tregs have been identified which may express different or different levels of particular markers. Tregs generally are T cells which express the markers CD4, CD25 and FOXP3 (CD4⁺CD25⁺FOXP3⁺). “FOXP3” is the abbreviated name of the forkhead box P3 protein. FOXP3 is a member of the FOX protein family of transcription factors and functions as a master regulator of the regulatory pathway in the development and function of regulatory T cells.

Tregs may also express CTLA-4 (cytotoxic T-lymphocyte associated molecule-4) or GITR (glucocorticoid-induced TNF receptor).

A Treg may be identified using the cell surface markers CD4 and CD25 in the absence of or in combination with low-level expression of the surface protein CD127 (CD4⁺CD25⁺CD127⁻ or CD4⁺CD25⁺CD127^{low}). The use of such markers to identify Tregs is known in the art and described in Liu et al. (JEM; 2006; 203; 7(10); 1701-1711), for example.

A Treg may be a CD4⁺CD25⁺FOXP3⁺ T cell, a CD4⁺CD25⁺CD127⁻ T cell, or a CD4⁺CD25⁺FOXP3⁺CD127^{-/low} T cell.

A Treg may have a demethylated Treg-specific demethylated region (TSDR). The TSDR is an important methylation-sensitive element regulating Foxp3 expression (Polansky, J.K., et al., 2008. European journal of immunology, 38(6), pp.1654-1663).

Different subpopulations of Tregs are known to exist, including naïve Tregs (CD45RA⁺FoxP3^{low}), effector/memory Tregs (CD45RA⁻FoxP3^{high} ) and cytokine-producing Tregs (CD45RA⁻FoxP3^{low}). “Memory Tregs” are Tregs which express CD45RO and which are considered to be CD45RO⁺. These cells have increased levels of CD45RO as compared to naïve Tregs (e.g. at least 10, 20, 30, 40, 50, 60, 70, 80 or 90% more CD45RO) and which preferably do not express or have low levels of CD45RA (mRNA and/or protein) as compared to naïve Tregs (e.g. at least 80, 90 or 95% less CD45RA as compared to naïve Tregs). “Cytokine-producing Tregs” are Tregs which do not express or have very low levels of CD45RA (mRNA and/or protein) as compared to naïve Tregs (e.g. at least 80, 90 or 95% less CD45RA as compared to naïve Tregs), and which have low levels of FOXP3 as compared to Memory Tregs, e.g. less than 50, 60, 70, 80 or 90% of the FOXP3 as compared to Memory Tregs. Cytokine-producing Tregs may produce interferon gamma and may be less suppressive in vitro as compared to naïve Tregs (e.g. less than 50, 60, 70, 80 or 90% suppressive than naïve Tregs. Reference to expression levels herein may refer to mRNA or protein expression. Particularly, for cell surface markers such as CD45RA, CD25, CD4, CD45RO etc, expression may refer to cell surface expression, i.e. the amount or relative amount of a marker protein that is expressed on the cell surface. Expression levels may be determined by any known method of the art. For example, mRNA expression levels may be determined by Northern blotting/array analysis, and protein expression may be determined by Western blotting, or preferably by FACS using antibody staining for cell surface expression.

Particularly, the Treg may be a naïve Treg. “A naïve regulatory T cell, a naïve T regulatory cell, or a naïve Treg” as used interchangeably herein refers to a Treg cell which expresses CD45RA (particularly which expresses CD45RA on the cell surface). Naïve Tregs are thus described as CD45RA⁺. Naïve Tregs generally represent Tregs which have not been activated through their endogenous TCRs by peptide/MHC, whereas effector/memory Tregs relate to Tregs which have been activated by stimulation through their endogenous TCRs. Typically, a naïve Treg may express at least 10, 20, 30, 40, 50, 60, 70, 80 or 90% more CD45RA than a Treg cell which is not naïve (e.g. a memory Treg cell). Alternatively viewed, a naïve Treg cell may express at least 2, 3, 4, 5, 10, 50 or 100-fold the amount of CD45RA as compared to a non-naïve Treg cell (e.g. a memory Treg cell). The level of expression of CD45RA can be readily determined by methods of the art, e.g. by flow cytometry using commercially available antibodies. Typically, non-naïve Treg cells do not express CD45RA or low levels of CD45RA.

Particularly, naïve Tregs may not express CD45RO, and may be considered to be CD45RO⁻. Thus, naïve Tregs may express at least 10, 20, 30, 40, 50, 60, 70, 80 or 90% less CD45RO as compared to a memory Treg, or alternatively viewed at least 2, 3, 4, 5, 10, 50 or 100 fold less CD45RO than a memory Treg cell.

Although naïve Tregs express CD25 as discussed above, CD25 expression levels may be lower than expression levels in memory Tregs, depending on the origin of the naïve Tregs. For example, for naïve Tregs isolated from peripheral blood, expression levels of CD25 may be at least 10, 20, 30, 40, 50, 60, 70, 80 or 90% lower than memory Tregs. Such naïve Tregs may be considered to express intermediate to low levels of CD25. However, a skilled person will appreciate that naïve Tregs isolated from cord blood may not show this difference.

Typically, a naïve Treg as defined herein may be CD4⁺, CD25⁺, FOXP3⁺, CD127^{low}, CD45RA⁺.

Low expression of CD127 as used herein refers to a lower level of expression of CD127 as compared to a CD4⁺ non-regulatory or Tcon cell from the same subject or donor. Particularly, naïve Tregs may express less than 90, 80, 70, 60, 50, 40, 30, 20 or 10% CD127 as compared to a CD4⁺ non-regulatory or Tcon cell from the same subject or donor. Levels of CD127 can be assessed by methods standard in the art, including by flow cytometry of cells stained with an anti-CD127 antibody.

Typically, naïve Tregs do not express, or express low levels of CCR4, HLA-DR, CXCR3 and/or CCR6. Particularly, naïve Tregs may express lower levels of CCR4, HLA-DR, CXCR3 and CCR6 than memory Tregs, e.g. at least 10, 20, 30, 40, 50, 60, 70, 80 or 90% lower level of expression.

Naïve Tregs may further express additional markers, including CCR7⁺ and CD31⁺.

Isolated naïve Tregs may be identified by methods known in the art, including by determining the presence or absence of a panel of any one or more of the markers discussed above, on the cell surface of the isolated cells. For example, CD45RA, CD4, CD25 and CD127 low can be used to determine whether a cell is a naïve Treg. Methods of determining whether isolated cells are naïve Tregs or have a desired phenotype can be carried out as discussed below in relation to additional steps which may be carried out as part of the invention, and methods for determining the presence and/or levels of expression of cell markers are well-known in the art and include, for example, flow cytometry, using commercially available antibodies.

The cell in which the polypeptide is to be expressed may be derived from a patient, that is from a subject to be treated. For example, the cell may have been removed from a subject and then transduced ex vivo with a vector, or other construct, according to the present invention. Alternatively, the cell may be a donor cell, for transfer to a recipient subject, or from a cell line., e.g. an NK cell line. The cell may further be a pluripotent cell (e.g. an iPSC) which may be differentiated to a desired target cell type, e.g. to a T cell, particularly to a Treg. Thus, the cell may autologous, syngeneic or allogeneic to the subject to be treated.

T cell populations which are suitable for ACT include: bulk peripheral blood mononuclear cells (PBMCs), CD8+ cells (for example, CD4-depleted PBMCs); PBMCs that are selectively depleted of T-regulatory cells (Tregs); isolated central memory (Tern) cells; EBV-specific CTLs; and tri-virus-specific CTLs and Treg cell preparations and populations as discussed above.

The present invention also comprises a cell population which comprises a cell according to the present invention. The cell population may have been transduced with a vector according to the present invention. A proportion of the cells of the cell population may express a polypeptide according to the first aspect of the invention at the cell surface. A proportion of the cells of the cell population may co-express a polypeptide according to the first aspect of the invention and a POI at the cell surface. The cell population may be an ex vivo patient-derived cell population.

The present invention also provides a method for deleting cells which express a polypeptide of the invention at the cell surface. The cell may be cell which comprises a nucleic acid molecule or vector or recombinant construct as defined or described herein, i.e. a cell into which the nucleic acid molecule or vector or construct has been introduced e.g. a cell transduced by a vector according to the present invention. The method comprises the step of exposing the cells to Rituximab, or an antibody having the binding specificity of Rituximab (i.e. an equivalent antibody).

Typically, Rituximab exerts its effects through complement-mediated cell killing, although other mechanisms may be involved, for example ADCC. Accordingly, in one embodiment the cells may be exposed to complement and Rituximab, or an equivalent antibody.

The method includes a method carried out in vitro to delete cells, e.g. in culture. However, the primary use is to delete cells in vivo, i.e. to delete cells which have previously been administered to a subject.

It will be appreciated that in vivo this may be achieved by administering the Rituximab or equivalent antibody to a subject to whom the cell has previously been administered, in other words a subject who has previously received ACT with a cell of the invention, which expresses the polypeptide. Complement may be present endogenously in the subject.

Thus, according to the present invention, Rituximab, or an antibody having the binding specificity thereof, may be provided for use in ACT in combination with a cell of the invention. As noted above the cell or nucleic acid or vector or construct for production of the cell and the Rituximab or equivalent antibody may be provided in a kit, or as a combination product.

When the polypeptide of the invention is expressed at the surface of a cell, binding of Rituximab or equivalent antibody to the R epitopes of the polypeptide causes lysis of the cell.

The term “delete” as used herein is synonymous with “remove” or “ablate”. The term is used to encompass cell killing, or inhibition of cell proliferation, such that the number of cells in the subject may be reduced. 100% complete removal may be desirable, but may not necessarily be achieved. Reducing the number of cells, or inhibiting their proliferation, in the subject may be sufficient to have a beneficial effect.

An antibody which has the binding specificity of Rituximab is an antibody which is capable of binding to the same natural epitope as does Rituximab. In particular, the antibody is capable of binding to the epitopes R1 and R2.

An antibody having the binding specificity of Rituximab may comprise an antigen binding domain of or from Rituximab. More particularly, it may comprise a VL and a VH domain from Rituximab, or the CDRs of Rituximab. Further, the antigen binding domain of Rituximab may be modified (e.g. by amino acid substitution, deletion or insertion) as long as the binding specificity of Rituximab is retained.

As noted above, biosimilars for Rituximab are available and may be used. A person of skill in the art is readily able to use routine methods to prepare an antibody having the binding specificity of Rituximab using the available amino acid sequences therefor.

In an embodiment the antibody having the binding specificity of Rituximab is in the conventional immunoglobulin format. That is it may comprise light and heavy chains and both constant and variable regions. The antibody may be bivalent, that is it may comprise two antigen binding sites. Other antibody formats may also be used, including for example a single chain format, or a monovalent format. The antibody may thus be of any class or type, or format,

More than one molecule of Rituximab or equivalent antibody may bind per polypeptide expressed at the cell surface. Each R epitope of the polypeptide may bind a separate molecule of Rituximab or equivalent antibody.

The decision to delete the transferred cells may arise from undesirable effects being detected in the subject which are attributable to the transferred cells. For example, unacceptable levels of toxicity may be detected.

CD20-expressing cells may be selectively ablated by treatment with the antibody Rituximab. As CD20 expression is absent from plasma cells, humoral immunity is retained following Rituximab treatment despite deletion of the B-cell compartment.

Adoptive transfer of genetically modified immune cells such as T cells is an attractive approach for generating desirable immune responses, such as an anti-tumour immune response, or to suppress or prevent an unwanted immune response.

The present invention provides a method for treating and/or preventing a disease or condition in a subject, which comprises the step of administering a cell according to the invention to the subject. The method may comprise the step of administering a population of cells to a subject.

The method may involve the following steps:
(i) taking a sample of cells, such as a blood sample from a patient or from a donor,
(ii) extracting the immune cell, e.g. T-cells,
(iii) introducing into the cells (e.g. transducing or transfecting the cells with) a vector or construct of the present invention which comprises a nucleic acid molecule encoding polypeptide and optionally a transgene of interest,
(iv) expanding the cells comprising the vector or construct (i.e. the modified cells) ex-vivo,
(v) administering the cells to the subject.

The modified cells may possess a desired therapeutic property such as enhanced tumour specific targeting and killing or immunosuppressive activity. It will be appreciated by a skilled person that the cells may be allogenic or autologous to the subject to be treated.

The cells of the present invention may be used to treat a cancer. Virtually all tumours are susceptible to lysis using an ACT approach and all are able to stimulate cytokine release from anti-tumour lymphocytes when tumour antigen is encountered.

The cells of the present invention may, for example, may be used to treat lymphoma, B-lineage malignancies, metastatic renal cell carcinoma (RCC), metastatic melanoma or neuroblastoma.

Alternatively, the cells of the invention may be used to treat or prevent a non-cancerous disease. The disease may be an infectious disease or a condition associated with transplantation, or any other unwanted or harmful immune response. The cells may be used for immunosuppression, for example to induce tolerance or treat or prevent an autoimmune or allergic condition. Particularly, the cells may be used to treat a neurodegenerative condition, such as Alzheimer’s disease, Parkinson’s disease, Motor neurone disease etc, type I diabetes, multiple sclerosis, lupus (particularly SLE), or an inflammatory condition, such as inflammatory bowel disease.

The cells of the invention may be used to treat or prevent post-transplantation lymphoproliferative disease (PTLD) or GvHD, or to prevent transplant rejection, e.g. of liver or kidney.

The invention will now be further described by way of Examples, which are meant to serve to assist one of ordinary skill in the art in carrying out the invention and are not intended in any way to limit the scope of the invention.

### EXAMPLES

### Example 1

The different Rituximab-based safety switches were designed as shown in FIG. 1. The sequences presented in FIG. 1 are for the R1-L-R2 part of the polypeptides only; the stalk sequences and signal peptide (leader) sequences are not shown. The depicted RQR8, SGGGGS-CD8a, CD8a, 1xSGGGGS and 3xSGGGGS sequences are SEQ ID NOS. 59, 60, 61, 62, and 63 respectively.

Switches 1xSGGGGS and 3xSGGGGS correspond to the polypeptides of SEQ ID NOS. 55 and 56 respectively, comprising the polypeptides of SEQ ID NOS, 27 and 28 respectively with the signal peptide (leader sequence) of SEQ ID NO. 54. Alternatively, as noted above, SEQ ID NOS 55 and 56 respectively may be seen as comprising the polypeptides of SEQ ID NOS, 78 and 79 respectively with the signal peptide (leader sequence) of SEQ ID NO. 80.

The switches were all expressed with the leader sequence of SEQ ID NO. 54 or SEQ ID NO.80 and the stalk sequence of SEQ ID NO. 26 with a linker of SEQ ID NO. 53.

The full amino acid sequences of RQR8, SGGGGS-CD8a, and CD8a are shown in SEQ ID NOS. 75, 76, and 77 respectively.

SEQ ID NO.57 shows the DNA sequence for 1xSGGGGS and SEQ ID NO. 58 shows the DNA sequence for 3xSGGGGS as used in this Example.

Switches (polypeptides) identified as SGGGGS-CD8a (SEQ ID NO. 60), 1xSGGGGS (SEQ ID 62) and 3xSGGGGS (SEQ ID NO. 63) have flexible linkers according to the invention herein. RQR8 of EP2836511 (SEQ ID NO. 59) and CD8A (SEQ ID NO. 61) are included for comparison.

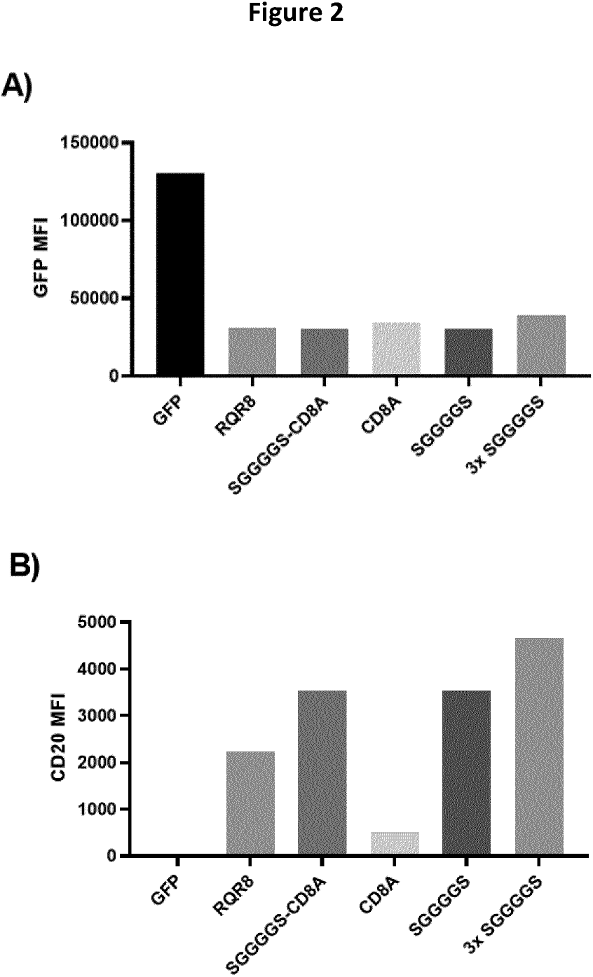

The safety switches were cloned into a lentiviral expression vector, linked to an eGFP protein via an 2A linker sequence. Jurkat cells were transduced with the different constructs and eGFP expression was assessed by flow cytometry (FIG. 2A). In a next step, cells were stained with a Rituximab Biosimilar antibody, conjugated to Alexa-Fluor 647 (clone HU2, R&D Systems). Staining efficiency was assessed as median fluorescence intensity (MFI) for GFP+ cells (FIG. 2B).

It can be seen that all the switches were expressed on the surface of the cells, and comparable levels of eGFP expression were seen (FIG. 2A). Switches SGGGGS-CD8a, 1xSGGGGS and 3xSGGGGS showed superior cell surface expression, as compared to RQR8, as can be seen from the detected expression of CD20 epitopes bound by the Rituximab Biosimilar antibody. CD8A, which does not have a flexible linker, exhibited poor CD20 epitope expression on the cell surface. Switch 3xSGGGGS was particularly well expressed.

### Example 2

Next, complement mediated killing (CMC) was assessed on cells, transduced with the different safety switch constructs. To this end, cells were cultured in the presence of baby rabbit complement, Rituximab and baby rabbit complement or RPMI cell culture medium alone. After 4 hours of culture killing efficiency was assessed by flow cytometry. Here, remaining percentage of GFP-positive cells was assessed.

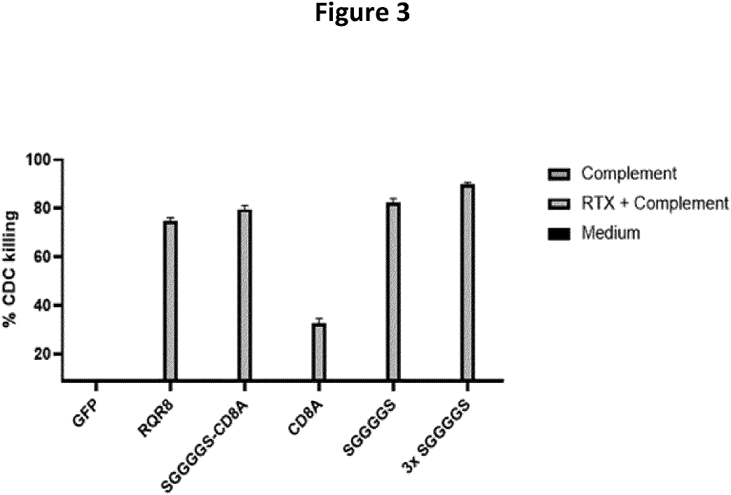

FIG. 3 shows that all the switches exhibited CMC, but that with switch CD8A was much lower than with the others. Switches SGGGGS-CD8a, 1xSGGGGS and 3xSGGGGS exhibited improved CMC as compared to RQR8, particularly 3xSGGGGS.

### Example 3

The sensitivity of the various safety switches was examined using Rituximab serial dilutions in a complement-dependent cytotoxicity (CDC) assay.

Firstly, the expression of the safety switches RQR8 (SEQ ID NO. 75), 1xSGGGGS (also referred to as RR8 small; SEQ ID NO. 55) and 3xSGGGGS (also referred to as RR8 large; SEQ ID NO. 56) was compared, as described in Example 1.

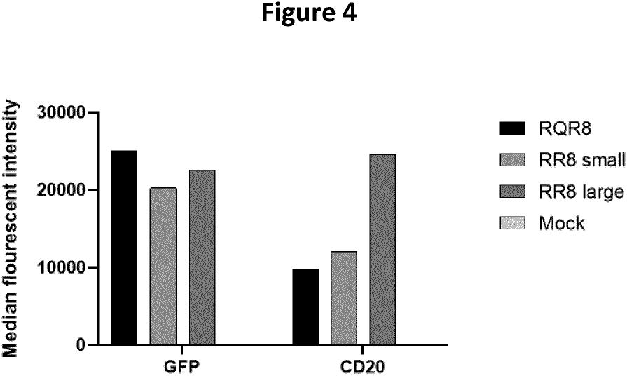

The safety switches were cloned into a lentiviral expression vector, linked to an eGFP protein via an 2A linker sequence. Jurkat cells were transduced with the different constructs and eGFP expression was assessed by flow cytometry (FIG. 4). In a next step, cells were stained with Rituximab antibody, conjugated to a flurorohore). Staining efficiency was assessed as median fluorescence intensity (MFI) for GFP+ cells (FIG. 4).

It can be seen that all three switches were expressed on the surface of the cells, and roughly similar levels of eGFP expression were seen. (FIG. 4). Switch 1xSGGGGS (RR8 small) showed slightly greater expression than RQR8, and 3xSGGGGS (RR8 large) showed superior cell surface expression, as compared to RQR8 , as can be seen from the detected expression of CD20 epitopes bound by the Rituximab antibody (FIG. 4). This confirms the previously seen result that Switch 3xSGGGGS (RR8 large) was particularly well expressed.

Rituximab serial dilutions were prepared in baby rabbit complement. Jurkat cells transduced with lentiviral vectors encoding SEQ ID NO. 75 (RQR8), SEQ ID NO. 55 (1x SGGGGS) or SEQ ID NO. 56 (3X SGGGGS) (as in Example 1), were counted and resuspended in RPMI. Cells were added in triplicate or duplicate per condition, to a 96 well plate (100,000 Jurkats per well). 50 uL of each Rituximab dilution (final concentrations: 100 ug/ml, 5 ug/ml, 2.5 ug/ml, 1.25 ug/ml, 0.625 ug/ml) (final volume of baby rabbit complement 50%) were added to each well of the 96 well plate, with a media alone condition and a complement alone condition. Plates were left to incubate, and after incubation the plate was stained for viability (Live/Dead NIR kit) and QBEND and analysed by FACS.

### Results

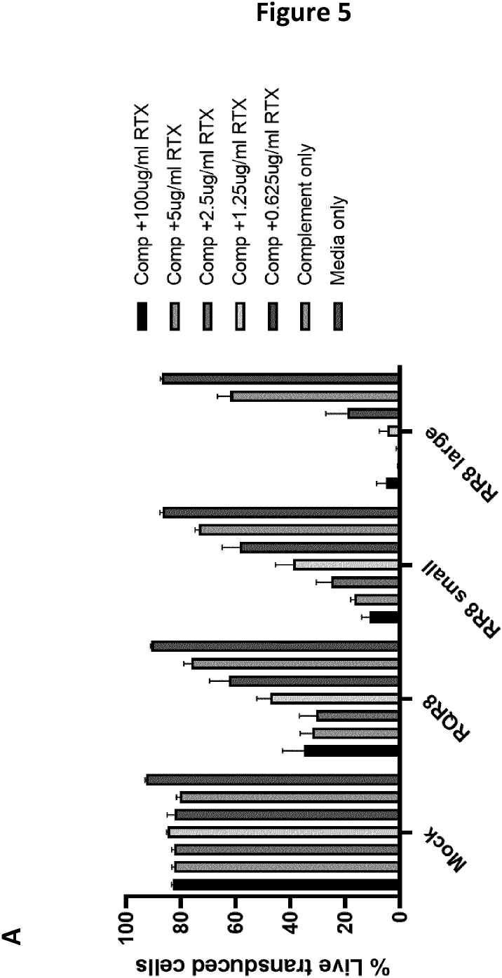

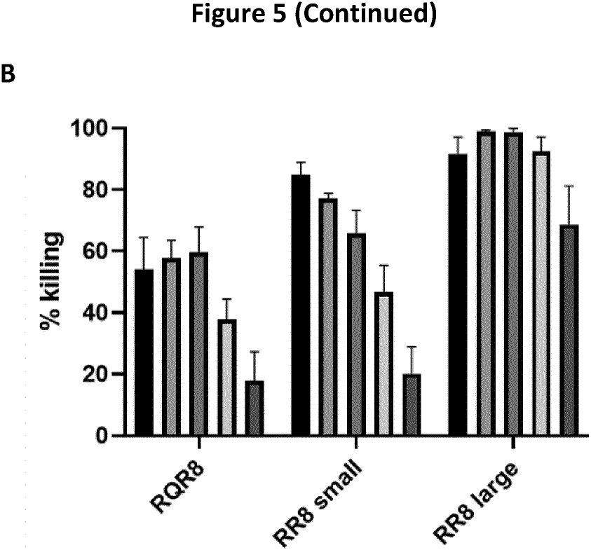

The results as presented in FIG. 5A show that the media and complement alone conditions did not result in significant cell death for Jurkat cells expressing any of the safety switches of SEQ ID NOS. 75, 55 or 56. However, upon addition of Rituximab, cells expressing SEQ ID NOS. 55 (RR8 small) and 56 (RR8 large) experienced significant killing across the range of Rituximab concentrations, with the highest level of killing being observed at the highest concentration of Rituximab, but with cell death levels at a high level (or alternatively viewed % live cells at a low level) even at the lowest Rituximab concentrations (FIGS. 5A and 5B). In contrast to this, cells expressing SEQ ID NO. 75 (RQR8) did not appear to be as sensitive to Rituximab as cells expressing SEQ ID NOS. 55 or 56 - % live transduced cells present after Rituximab treatment was much higher for SEQ ID NO. 75 expressing cells across all Rituximab concentrations. Thus, surprisingly, and advantageously, the safety switches having amino acid sequences SEQ ID NOS. 55 and 56 appear to be more effective and sensitive than RQR8, potentially requiring less antibody to induce cell death.The RQR8 (SEQ ID NO. 75) and RR8 small linker (SEQ ID NO. 55) have very similar GFP and CD20 MFIs, so these results can be directly compared. As can be seen from FIG. 5B, the RR8 small shows a nice dose dependent killing response, with similar if not better ability to kill cells at the highest concentration of RTX. The RR8 large linker had a significantly higher level of CD20 MFI and the results reflect this, with a high level of killing even at the lowest concentration.

It is noted also that it can be seen in FIG. 5 that for both RQR8 and RR8 large, the highest concentration of Rituximab, resulted in a higher percentage of live cells compared to the next lower concentrations. This may indicate the killing has reached capacity.

The present inventors have designed new safety switches which may be used in cells for ACT. The translated protein is stably expressed on the cell surface after retroviral transduction. The construct binds Rituximab, and the dual epitope design engenders highly effect complement mediated killing. Due to the small size of the construct, it can easily be co-expressed with typical T-cell engineering transgenes such as T-cell receptors or Chimeric Antigen Receptors and others allowing deletion of cells in the face of unacceptable toxicity with off the shelf clinical-grade reagents / pharmaceuticals.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in cell therapy, T-cell engineering, molecular biology or related fields are intended to be within the scope of the following claims.

1. A polypeptide comprising a sequence having the formula:
R1-L-R2-St wherein
R1 and R2 are Rituximab-binding epitopes;
St is a stalk sequence which, when the polypeptide is expressed at the surface of a target cell, causes the R1 and R2 epitopes to be projected from the cell surface; and
L is a flexible linker sequence which connects the C terminus of R1 to the N terminus of R2 and which does not comprise a QBEnd10 binding epitope comprising the sequence set out in SEQ ID NO.1.

2. The polypeptide of claim 1 wherein L is selected from:
(i) a flexible linker sequence having a length of no more than 25, preferably no more than 24, 23, 22 or 21 amino acids; and/or
(ii) a linker sequence which comprises at least 40% Gly or Gly and Ser residues; and/or
(iii) a linker sequence comprising Ser and/or Gly residues, and no more than 15 other amino acid residues, preferably no more than 14, 13, 12, 11, 10, 9, 8, 6, 7, 5, or 4 other amino acid residues; and/or
(iv) a linker sequence having an amino acid sequence wherein at least 80%, 90% or 100% of the amino acid residues are Ser, Gly, Thr, Ala, Lys, and Glu residues; and/or
(v) a linker sequence having an amino acid sequence which does not comprise any Pro residues.

3. The polypeptide of claim 1 or claim 2, wherein L does not comprise a marker sequence.

4. The polypeptide of any one of claims 1 to 3, wherein the linker sequence L comprises at least one Gly-Ser domain composed solely of Ser and Gly residues, and no more than 15 other amino acid residues, preferably no more than 14, 13, 12, 11, 10, 9, 8, 6, 7, 5, or 4 other amino acid residues.

5. The polypeptide of claim 4, wherein the Gly-Ser domain has the formula:
(S)q-[(G)m-(S)m]n-(G)p
wherein q is 0 or 1; m is an integer from 1-8; n is an integer of at least 1 (e.g. from 1 to 8, or preferably 1 to 6); and p is 0 or an integer from 1 to 3.

6. The polypeptide of claim 5, wherein the Gly-Ser domain has the formula:
(i) S-[(G)m-S]n;
(ii) [(G)m-S]n; or
(iii) [(G)m-S]n-(G)p
wherein m is an integer from 2-8 (preferably 3-4); n is an integer of at least 1 (e.g. from 1 to 8, or preferably 1 to 6); and p is 0 or an integer from 1 to 3.

7. The polypeptide of any one of claims 4 to 6, wherein the Gly-Ser domain has the formula:
S-[G-G-G-G-S]n
wherein n is an integer of at least one (preferably 1 to 8, or 1-6, 1-5, 1-4, or 1-3).

8. The polypeptide of any one of claims 1 to 7, wherein the linker sequence is selected from:
ETSGGGGSRL (SEQ ID NO. 32)
SGGGGSGGGGSGGGGS ((SEQ ID NO. 33)
S(GGGGS)₁₋₅ (where GGGGS is SEQ ID NO. 31)
(GGGGS)₁₋₅ (where GGGGS is SEQ ID NO. 31)
S(GGGS)₁₋₅ (where GGGS is SEQ ID NO. 34)
(GGGS)₁₋₅ (where GGGS is SEQ ID NO. 34)
S(GGGGGS)₁₋₅ (where GGGGGS is SEQ ID NO. 35)
(GGGGGS)₁₋₅ (where GGGGGS is SEQ ID NO. 35)
S(GGGGGGS)₁₋₅ (where GGGGGGS is SEQ ID NO. 36)
(GGGGGGS)₁₋₅ (where GGGGGGS is SEQ ID NO. 36)
G₆ (SEQ ID NO. 37)
G₈ (SEQ ID NO. 38)
KESGSVSSEQLAQFRSLD (SEQ ID NO.39)
EGKSSGSGSESKST (SEQ ID NO.40)
GSAGSAAGSGEF (SEQ ID NO.41)
SGGGGSAGSAAGSGEF (SEQ ID NO.42)
SGGGLLLLLLLLGGGS (SEQ ID NO.43)
SGGGAAAAAAAAGGGS (SEQ ID NO.44)
SGGGAAAAAAAAAAAAAAAAGGGS (SEQ ID NO.45)
SGALGGLALAGLLLAGLGLGAAGS (SEQ ID NO.46)
SLSLSPGGGGGPAR (SEQ ID NO.47)
SLSLSPGGGGGPARSLSLSPGGGGG (SEQ ID NO.48)
GSSGSS (SEQ ID NO.49)
GSSSSSS (SEQ ID NO.50)
GGSSSS (SEQ ID NO.51)
GSSSSS (SEQ ID NO.52)
SGGGGS (SEQ ID NO. 53.

9. The polypeptide of any one of claims 1 to 8, wherein the Rituximab-binding epitopes R1 and R2 each comprise:
(a) an amino acid sequence of the consensus sequence X1-C-X2-X3-(A/S)-N-P-S-X4-C (SEQ ID NO. 2), wherein X1 is A or absent, and X2, X3 and X4 are any amino acid; or
(b) an amino acid sequence as set out in SEQ ID NO. 3 or a variant thereof having at least 75% sequence identity thereto and which retains Rituximab-binding activity.

10. The polypeptide of claim 9, wherein in the consensus sequence X1-C-X2-X3-(A/S)-N-P-S-X4-C (SEQ ID NO. 1) for the Rituximab-binding epitopes R1 and R2, X2 is P, N, S,M, W or E; X3 is Y, F, W,A, or H; and X4 is L, T, M or Q.

11. The polypeptide of any one of claims 1 to 10, wherein the Rituximab-binding epitopes R1 and R2 each comprise an amino acid sequence as set out in any one of SEQ ID NO.s 4 to 14 or 15 to 25, or a variant thereof having at least 75% sequence identity thereto and which retains Rituximab-binding activity.

12. The polypeptide of any one of claims 1 to 11, wherein the stalk sequence St comprises an optional linker sequence which connects it to R2, an extracellular domain, an optional transmembrane domain, and an optional intracellular domain.

13. The polypeptide of claim 12, wherein the stalk sequence St comprises a linker sequence which connects it to R2, an extracellular domain, a transmembrane domain, and an intracellular domain.

14. The polypeptide of claim 12 or claim 13, wherein the stalk sequence St comprises an extracellular domain which is derived from the extracellular stalk sequence of a protein selected from CD27, CD28, CD3 epsilon, CD3z, CD45, CD4, CD5, CD8, CD9, CD16, CD18, CD22, CD33, CD37, CD64, CD80, CD86, CD134, CD137, CD152, CD154, CD278, CD279, IgG1, or IgG2, optionally together with (i) a transmembrane domain or (ii) a transmembrane domain and an intracellular domain derived from an aforesaid protein, wherein the extracellular stalk sequence, transmembrane domain and intracellular domain may be from the same or different proteins.

15. The polypeptide of any one of claims 1 to 14, wherein the stalk sequence St comprises an extracellular stalk sequence, a transmembrane domain, and an intracellular domain derived from CD8.

16. The polypeptide of claim 15, wherein the stalk sequence St comprises the amino acid sequence set out in SEQ ID NO. 26, or a sequence having at least 80% sequence identity thereto.

17. The polypeptide of any one of claims 1 to 16, which comprises the sequence set out in SEQ ID NO. 27, 28, 78 or 79, or a sequence having at least 75% identity thereto which (i) binds Rituximab and (ii) when expressed on the surface of a cell, induces killing of the cell in the presence of Rituximab.

18. A fusion protein which comprises a polypeptide as defined in any one of claims 1 to 17 linked to a polypeptide fusion partner, optionally via a linker sequence.

19. The fusion protein of claim 18, wherein the fusion partner is a polypeptide comprising a marker sequence, or a chimeric receptor, preferably a chimeric antigen receptor (CAR), or a T cell receptor (TCR).

20. The fusion protein of claim 18 or 19, wherein the fusion protein comprises a self-cleaving peptide between the polypeptide and a chimeric receptor or TCR.

21. A nucleic acid molecule comprising a nucleotide sequence which encodes the polypeptide of any one of claims 1 to 17 or the fusion protein of any one of claims 18 to 20.

22. A vector which comprises a nucleic acid molecule according to claim 21.

23. A vector according to claim 22, which also comprises a transgene of interest, preferably which encodes a protein of interest (POI).

24. A vector according to claim 23, wherein the transgene of interest encodes an antigen receptor (e.g. a chimeric receptor, preferably a chimeric antigen receptor (CAR), or a T-cell receptor), such that when the vector is introduced into a target cell, the target cell co-expresses a polypeptide according to any of claims 1 to 15 and the antigen receptor.

25. A cell which expresses a polypeptide according to any of claims 1 to 17.

26. The cell of claim 25, wherein the cell co-expresses the polypeptide and a POI at the cell surface.

27. A cell which comprises a nucleic acid molecule according to claim 21 or a vector according to any one of claims 22 to 24.

28. The cell of any one of claims 25 to 27, which is a T cell, preferably a Treg cell.

29. A method for making a cell according to any of claims 25 to 28, which comprises the step of introducing into the cell (e.g. transducing or transfecting a cell with) a vector according to any of claims 22 to 24.

30. A method for deleting a cell according to any of claims 25 to 28, which comprises the step of exposing the cell to an antibody having the binding specificity of Rituximab.

31. A method for treating a disease in a subject, which comprises the step of administering a cell according to any of claims 25 to 28 to the subject.

32. The method of claim 31, which comprises the following steps:
(i) introducing into a sample of cells (e.g. transducing or transfecting the cells with) a vector according to any one of claims 22 to 24, and
(ii) administering the cells to the subject, optionally wherein the cells are isolated from the subject and are returned to the subject in step (ii).

33. A cell according to any one of claims 25 to 28 for use in adoptive cell transfer therapy.

34. A method according to any one of claims 31 or 32, or the cell for use according to claim 33, for treating cancer, an infectious, neurodegenerative or inflammatory disease, or for inducing immunosuppression.


SEQUENCE LISTING (80 sequences)

SEQ ID NO: 1 - protein, 16 aa, Artificial Sequence
  QBEnd10 binding epitope
ELPTQGTFSNVSTNVS

SEQ ID NO: 2 - protein, 11 aa, Artificial Sequence
  Consensus sequence for Rituximab mimotope
  misc_feature (1)..(1): Xaa can be any naturally occurring amino acid
  misc_feature (3)..(4): Xaa can be any naturally occurring amino acid
  misc_feature (10)..(10): Xaa can be any naturally occurring amino acid
XCXXASNPSXC

SEQ ID NO: 3 - protein, 12 aa, Artificial Sequence
  Rituximab mimetope
QDKLTQWPKWLE

SEQ ID NO: 4 - protein, 9 aa, Artificial Sequence
  Modified Rituximab mimetope
CPYANPSLC

SEQ ID NO: 5 - protein, 9 aa, Artificial Sequence
  Modified Rituximab mimetope
CPYSNPSLC

SEQ ID NO: 6 - protein, 9 aa, Artificial Sequence
  Modified Rituximab mimetope
CPFANPSTC

SEQ ID NO: 7 - protein, 9 aa, Artificial Sequence
  Modified Rituximab mimetope
CNFSNPSLC

SEQ ID NO: 8 - protein, 9 aa, Artificial Sequence
  Modified Rituximab mimetope
CPFSNPSMC

SEQ ID NO: 9 - protein, 9 aa, Artificial Sequence
  Modified Rituximab mimetope
CSWANPSQC

SEQ ID NO: 10 - protein, 9 aa, Artificial Sequence
  Modified Rituximab mimetope
CMFSNPSLC

SEQ ID NO: 11 - protein, 9 aa, Artificial Sequence
  Modified Rituximab mimetope
CPFANPSMC

SEQ ID NO: 12 - protein, 9 aa, Artificial Sequence
  Modified Rituximab mimetope
CWASNPSLC

SEQ ID NO: 13 - protein, 9 aa, Artificial Sequence
  Modified Rituximab mimetope
CEHSNPSLC

SEQ ID NO: 14 - protein, 9 aa, Artificial Sequence
  Modified Rituximab mimetope
CWAANPSMC

SEQ ID NO: 15 - protein, 10 aa, Artificial Sequence
  Rituximab mimetope
ACPYANPSLC

SEQ ID NO: 16 - protein, 10 aa, Artificial Sequence
  Rituximab mimetope
ACPYSNPSLC

SEQ ID NO: 17 - protein, 10 aa, Artificial Sequence
  Rituximab mimetope
ACPFANPSTC

SEQ ID NO: 18 - protein, 10 aa, Artificial Sequence
  Rituximab mimetope (R8-, R12-, R18-C)
ACNFSNPSLC

SEQ ID NO: 19 - protein, 10 aa, Artificial Sequence
  Rituximab mimetope (R14-C)
ACPFSNPSMC

SEQ ID NO: 20 - protein, 10 aa, Artificial Sequence
  Rituximab mimetope
ACSWANPSQC

SEQ ID NO: 21 - protein, 10 aa, Artificial Sequence
  Rituximab mimetope
ACMFSNPSLC

SEQ ID NO: 22 - protein, 10 aa, Artificial Sequence
  Rituximab mimetope
ACPFANPSMC

SEQ ID NO: 23 - protein, 10 aa, Artificial Sequence
  Rituximab mimetope
ACWASNPSLC

SEQ ID NO: 24 - protein, 10 aa, Artificial Sequence
  Rituximab mimetope
ACEHSNPSLC

SEQ ID NO: 25 - protein, 10 aa, Artificial Sequence
  Rituximab mimetope
ACWAANPSMC

SEQ ID NO: 26 - protein, 82 aa, Artificial Sequence
  CD8 stalk sequence
PAPRPPTPAPTIASQPLSLRPEACRPAAGGAVHTRGLDFACDIYIWAPLAGTCGVLLLSLVITLYCNHRNRRRVCKCPRPVV

SEQ ID NO: 27 - protein, 116 aa, Artificial Sequence
  Polypeptide sequence
CPYSNPSLCETSGGGGSRLCPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 28 - protein, 122 aa, Artificial Sequence
  Polypeptide sequence
CPYSNPSLCSGGGGSGGGGSGGGGSCPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 29 - protein, 17 aa, Artificial Sequence
  CD20 Rituximab-binding epitope
CEPANPSEKNSPSTQYC

SEQ ID NO: 30 - protein, 42 aa, Artificial Sequence
  CD8 extracellular sequence
PAPRPPTPAPTIASQPLSLRPEACRPAAGGAVHTRGLDFACD

SEQ ID NO: 31 - protein, 5 aa, Artificial Sequence
  Representative exemplary linker sequence
GGGGS

SEQ ID NO: 32 - protein, 10 aa, Artificial Sequence
  Representative exemplary linker sequence
ETSGGGGSRL

SEQ ID NO: 33 - protein, 16 aa, Artificial Sequence
  Representative exemplary linker sequence
SGGGGSGGGGSGGGGS

SEQ ID NO: 34 - protein, 4 aa, Artificial Sequence
  Representative exemplary linker sequence
GGGS

SEQ ID NO: 35 - protein, 6 aa, Artificial Sequence
  Representative exemplary linker sequence
GGGGGS

SEQ ID NO: 36 - protein, 7 aa, Artificial Sequence
  Representative exemplary linker sequence
GGGGGGS

SEQ ID NO: 37 - protein, 6 aa, Artificial Sequence
  Representative exemplary linker sequence
GGGGGG

SEQ ID NO: 38 - protein, 8 aa, Artificial Sequence
  Representative exemplary linker sequence
GGGGGGGG

SEQ ID NO: 39 - protein, 18 aa, Artificial Sequence
  Representative exemplary linker sequence
KESGSVSSEQLAQFRSLD

SEQ ID NO: 40 - protein, 14 aa, Artificial Sequence
  Representative exemplary linker sequence
EGKSSGSGSESKST

SEQ ID NO: 41 - protein, 12 aa, Artificial Sequence
  Representative exemplary linker sequence
GSAGSAAGSGEF

SEQ ID NO: 42 - protein, 16 aa, Artificial Sequence
  Representative exemplary linker sequence
SGGGGSAGSAAGSGEF

SEQ ID NO: 43 - protein, 16 aa, Artificial Sequence
  Representative exemplary linker sequence
SGGGLLLLLLLLGGGS

SEQ ID NO: 44 - protein, 16 aa, Artificial Sequence
  Representative exemplary linker sequence
SGGGAAAAAAAAGGGS

SEQ ID NO: 45 - protein, 24 aa, Artificial Sequence
  Representative exemplary linker sequence
SGGGAAAAAAAAAAAAAAAAGGGS

SEQ ID NO: 46 - protein, 24 aa, Artificial Sequence
  Representative exemplary linker sequence
SGALGGLALAGLLLAGLGLGAAGS

SEQ ID NO: 47 - protein, 14 aa, Artificial Sequence
  Representative exemplary linker sequence
SLSLSPGGGGGPAR

SEQ ID NO: 48 - protein, 25 aa, Artificial Sequence
  Representative exemplary linker sequence
SLSLSPGGGGGPARSLSLSPGGGGG

SEQ ID NO: 49 - protein, 6 aa, Artificial Sequence
  Representative exemplary linker sequence
GSSGSS

SEQ ID NO: 50 - protein, 7 aa, Artificial Sequence
  Representative exemplary linker sequence
GSSSSSS

SEQ ID NO: 51 - protein, 6 aa, Artificial Sequence
  Representative exemplary linker sequence
GGSSSS

SEQ ID NO: 52 - protein, 6 aa, Artificial Sequence
  Representative exemplary linker sequence
GSSSSS

SEQ ID NO: 53 - protein, 6 aa, Artificial Sequence
  Representative exemplary linker sequence
SGGGGS

SEQ ID NO: 54 - protein, 21 aa, Artificial Sequence
  Signal peptide
MGTSLLCWMALCLLGADHADA

SEQ ID NO: 55 - protein, 137 aa, Artificial Sequence
  Polypeptide comprising the signal peptide of SEQ ID NO.54 and the amino acid sequence of SEQ ID NO. 27
MGTSLLCWMALCLLGADHADACPYSNPSLCETSGGGGSRLCPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 56 - protein, 143 aa, Artificial Sequence
  Polypeptide comprising the signal peptide of SEQ ID NO.54 and the amino acid sequence of SEQ ID NO. 28
MGTSLLCWMALCLLGADHADACPYSNPSLCSGGGGSGGGGSGGGGSCPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 57 - DNA, 410 bp, Artificial Sequence
  DNA for 1xSGGGGS as used in Example 1
tgggcacatctttgctttgttggatggccctgtgtctgctgggagccgatcatgctgatgcctgtccttacagcaaccccagcctgtgtgagacgagcggtggtggcggaagccgtctctgtccctactccaatcctagcctgtgtagcggaggtggcggaagccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgttctgctgctgagcctggtcatcaccctgtactgcaaccaccggaacaggcggagagtgtgcaagtgccctagacctgtggtg

SEQ ID NO: 58 - DNA, 429 bp, Artificial Sequence
  DNA for 3xSGGGGS as used in Example 1
atgggcacatctttgctttgttggatggccctgtgtctgctgggagccgatcatgctgatgcctgtccttacagcaaccccagcctgtgtagcggcggcggaggcagcggtggcggaggcagcggcggaggcggtagctgtccctactccaatcctagcctgtgtagcggaggtggcggaagccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgttctgctgctgagcctggtcatcaccctgtactgcaaccaccggaacaggcggagagtgtgcaagtgccctagacctgtggtg

SEQ ID NO: 59 - protein, 48 aa, Artificial Sequence
  RQR8
CPYSNPSLCSGGGGSELPTQGTFSNVSTNVSPAKPTTTACPYSNPSLC

SEQ ID NO: 60 - protein, 48 aa, Artificial Sequence
  SGGGGS-CD8A
CPYSNPSLCSGGGGSPAKPTTTPAPRPPTPAPTIASQPACPYSNPSLC

SEQ ID NO: 61 - protein, 34 aa, Artificial Sequence
  CD8A
CPYSNPSLCPAKPTTTPAPRPPTPACPYSNPSLC

SEQ ID NO: 62 - protein, 28 aa, Artificial Sequence
  1x SGGGGS
CPYSNPSLCETSGGGGSRLCPYSNPSLC

SEQ ID NO: 63 - protein, 34 aa, Artificial Sequence
  3x SGGGGS
CPYSNPSLCSGGGGSGGGGSGGGGSCPYSNPSLC

SEQ ID NO: 64 - protein, 20 aa, Artificial Sequence
  Linker sequence
SGGGSNVSTNVSPAKPTTTA

SEQ ID NO: 65 - protein, 20 aa, Artificial Sequence
  Linker sequence
SGGGSELPTQGTFSNVSTNA

SEQ ID NO: 66 - protein, 20 aa, Artificial Sequence
  Linker sequence
EAAAKEAAAKEAAAKEAAAK

SEQ ID NO: 67 - protein, 20 aa, Artificial Sequence
  Linker sequence
GGGGSEAAAKEAAAKSGGGS

SEQ ID NO: 68 - protein, 15 aa, Artificial Sequence
  Linker sequence
EAAAKEAAAKEAAAK

SEQ ID NO: 69 - protein, 16 aa, Artificial Sequence
  Linker sequence
GGLKNKAQQAAFYIGG

SEQ ID NO: 70 - protein, 14 aa, Artificial Sequence
  Linker sequence
LCKNKAQQAAFYCI

SEQ ID NO: 71 - protein, 14 aa, Artificial Sequence
  Linker sequence
KCLNDAQAAAEECI

SEQ ID NO: 72 - protein, 18 aa, Artificial Sequence
  Linker sequence
GGGLKNKAQQAAFYIGGG

SEQ ID NO: 73 - protein, 25 aa, Artificial Sequence
  Linker sequence
EAAAKEAAAKEAAAKEAAAEAAAKE

SEQ ID NO: 74 - protein, 24 aa, Artificial Sequence
  Linker sequence
GGGSEAAAKEAAAKEAAAKEGGGS

SEQ ID NO: 75 - protein, 157 aa, Artificial Sequence
  RQR8 as used in Example 1
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 76 - protein, 141 aa, Artificial Sequence
  SGGGGS-CD8A as used in Example 1
MGTSLLCWMALCLLGADHADACPYSNPSLCSGGGGSPAKPTTTPAPRPPTPAPTIASQPACPYSNPSLCSGGGGSLSLRPEACRPAAGGAVHTRGLDFACDIYIWAPLAGTCGVLLLSLVITLYCNHRNRRRVCKCPRPVV

SEQ ID NO: 77 - protein, 134 aa, Artificial Sequence
  CD8A as used in Example 1
MGTSLLCWMALCLLGADHADACPYSNPSLCPAKPTTTPAPRPPTPACPYSNPSLCSGGGGSPTIASQPLSLRPEACRPAAGGAVHTRGLDFACDIYIWAPLAGTCGVLLLSLVITLYCNHRNRRRVCKCPRPVV

SEQ ID NO: 78 - protein, 117 aa, Artificial Sequence
  Polypeptide sequence
ACPYSNPSLCETSGGGGSRLCPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 79 - protein, 123 aa, Artificial Sequence
  Polypeptide sequence
ACPYSNPSLCSGGGGSGGGGSGGGGSCPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 80 - protein, 20 aa, Artificial Sequence
  Signal peptide
MGTSLLCWMALCLLGADHAD